(19) United States
(12) Patent Application Publication
(10) Pub. No.: US 2006/0247491 A1
(43) Pub. Date: Nov. 2, 2006

(54) Devices and methods for heart valve treatment

(76) Inventors: Robert M. Vidlund, Maplewood, MN (US); Craig A. Ekvall, Elk River, MN (US); Jeffrey D. Santer, Spring Lake Park, MN (US); David A. Kusz, Minneapolis, MN (US); Cyril J. Schweich JR., Maple Grove, MN (US)

Correspondence Address: Susanne T. Jones;FINNEGAN, HENDERSON, FARABOW,, GARRETT & DUNNER, L.L.P., 901 New York Avenue, N.W., Washington, DC, 20001-4413, US

(21) Appl. No.: 11/115,409

(22) Filed: Apr. 27, 2005

(51) Int. Cl. A61F 2/02; A61F 2/24; A61B 17/34

(52) U.S. Cl. 600/37; 606/151; 606/139; 606/185; 623/2.37

(57) ABSTRACT

Devices and methods for improving the function of a valve (e.g., mitral valve) by positioning an implantable device outside and adjacent the heart wall such that the device alters the shape of the heart wall acting on the valve. The implantable device may alter the shape of the heart wall acting on the valve by applying an inward force and/or by circumferential shortening (cinching). The shape change of the heart wall acting on the valve is sufficient to change the function of the valve, and may increase coaptation of the leaflets, for example, to reduce regurgitation.

### CROSS REFERENCE TO RELATED APPLICATIONS

This patent application is related to U.S. patent application Ser. No. 10/704,143 filed Nov. 10, 2003 (published as US 2004/0148019 A1), and U.S. patent application Ser. No. 10/704,145 filed Nov. 10, 2003 (published as US 2004/0148020 A1), both of which claim the benefit of U.S. Provisional Patent Application No. 60/425,519, filed Nov. 12, 2002, all of which are entitled DEVICES AND METHODS FOR HEART VALVE TREATMENT to Vidlund et al., the entire disclosures of which are all incorporated herein by reference. This application also is related to U.S. patent application Ser. No. ______, filed on a date even herewith, entitled DEVICES AND METHODS FOR PERICARDIAL ACCESS to Vidlund et al. (Attorney Docket No. 07528.0047), the entire disclosure of which is incorporated herein by reference.

### FIELD OF THE INVENTION

The present invention relates to devices and associated methods for treating and improving the performance of dysfunctional heart valves. More particularly, the invention relates to devices and methods that passively assist to reshape a dysfunctional heart valve to improve its performance.

### BACKGROUND OF THE INVENTION

Various etiologies may result in heart valve insufficiency depending upon both the particular valve as well as the underlying disease state of the patient. For instance, a congenital defect may be present resulting in poor coaptation of the valve leaflets, such as in the case of a monocusp aortic valve, for example. Valve insufficiency also may result from an infection, such as rheumatic fever, for example, which may cause a degradation of the valve leaflets. Functional regurgitation also may be present. In such cases, the valve components may be normal pathologically, yet may be unable to function properly due to changes in the surrounding environment. Examples of such changes include geometric alterations of one or more heart chambers and/or decreases in myocardial contractility. In any case, the resultant volume overload that exists as a result of an insufficient valve may increase chamber wall stress. Such an increase in stress may eventually result in a dilatory process that further exacerbates valve dysfunction and degrades cardiac efficiency.

Mitral valve regurgitation often may be driven by the functional changes described above. Alterations in the geometric relationship between valvular components may occur for numerous reasons, including events ranging from focal myocardial infarction to global ischemia of the myocardial tissue. Idiopathic dilated cardiomyopathy also may drive the evolution of functional mitral regurgitation. These disease states often lead to dilatation of the left ventricle. Such dilatation may cause papillary muscle displacement and/or dilatation of the valve annulus. As the papillary muscles move away from the valve annulus, the chordae connecting the muscles to the leaflets may become tethered. Such tethering may restrict the leaflets from closing together, either symmetrically or asymmetrically, depending on the relative degree of displacement between the papillary muscles. Moreover, as the annulus dilates in response to chamber enlargement and increased wall stress, increases in annular area and changes in annular shape may increase the degree of valve insufficiency. Annular dilatation is typically concentrated on the posterior aspect, since this aspect is directly associated with the dilating left ventricular free wall and not directly attached to the fibrous skeleton of the heart. Annular dilatation also may result in a flattening of the valve annulus from its normal saddle shape.

Alterations in functional capacity also may cause valve insufficiency. In a normally functioning heart, the mitral valve annulus contracts during systole to assist in leaflet coaptation. Reductions in annular contractility commonly observed in ischemic or idiopathic cardiomyopathy patients therefore hamper the closure of the valve. Further, in a normal heart, the papillary muscles contract during the heart cycle to assist in maintaining proper valve function. Reductions in or failure of the papillary muscle function also may contribute to valve regurgitation. This may be caused by infarction at or near the papillary muscle, ischemia, or other causes, such as idiopathic dilated cardiomyopathy, for example.

The degree of valve regurgitation may vary, especially in the case of functional insufficiency. In earlier stages of the disease, the valve may be able to compensate for geometric and/or functional changes in a resting state. However, under higher loading resulting from an increase in output requirement, the valve may become incompetent. Such incompetence may only appear during intense exercise, or alternatively may be induced by far less of an exertion, such as walking up a flight of stairs, for example.

Conventional techniques for managing mitral valve dysfunction include either surgical repair or replacement of the valve or medical management of the patient. Medical management typically applies only to early stages of mitral valve dysfunction, during which levels of regurgitation are relatively low. Such medical management tends to focus on volume reductions, such as diuresis, for example, or afterload reducers, such as vasodilators, for example.

Early attempts to surgically treat mitral valve dysfunction focused on replacement technologies. In many of these cases, the importance of preserving the native subvalvular apparatus was not fully appreciated and many patients often acquired ventricular dysfunction or failure following the surgery. Though later experience was more successful, significant limitations to valve replacement still exist. For instance, in the case of mechanical prostheses, lifelong therapy with powerful anticoagulants may be required to mitigate the thromboembolic potential of these devices. In the case of biologically derived devices, in particular those used as mitral valve replacements, the long-term durability may be limited. Mineralization induced valve failure is common within ten years, even in younger patients. Thus, the use of such devices in younger patient groups is impractical.

Another commonly employed repair technique involves the use of annuloplasty rings. These rings originally were used to stabilize a complex valve repair. Now, they are more often used alone to improve mitral valve function. An annuloplasty ring has a diameter that is less than the diameter of the enlarged valve annulus. The ring is placed in the valve annulus and the tissue of the annulus sewn or otherwise secured to the ring. This causes a reduction in the annular circumference and an increase in the leaflet coaptation area. Such rings, however, generally flatten the natural saddle shape of the valve and hinder the natural contractility of the valve annulus. This may be true even when the rings have relatively high flexibility.

To further reduce the limitations of the therapies described above, purely surgical techniques for treating valve dysfunction have evolved. Among these surgical techniques is the Alfiere stitch or so-called bowtie repair. In this surgery, a suture is placed substantially centrally across the valve orifice joining the posterior and anterior leaflets to create leaflet apposition. Another surgical technique includes plication of the posterior annular space to reduce the cross-sectional area of the valve annulus. A limitation of each of these techniques is that they typically require opening the heart to gain direct access to the valve and the valve annulus. This generally necessitates the use of cardiopulmonary bypass, which may introduce additional morbidity and mortality to the surgical procedures. Additionally, for each of these procedures, it is very difficult to evaluate the efficacy of the repair prior to the conclusion of the operation.

Due to these drawbacks, devising effective techniques that could improve valve function without the need for cardiopulmonary bypass and without requiring major remodeling of the valve may be advantageous. In particular, passive techniques to change the shape of the heart chamber and/or associated valve and reduce regurgitation while maintaining substantially normal leaflet motion may be desirable. Further, advantages may be obtained by a technique that reduces the overall time a patient is in surgery and under the influence of anesthesia. It also may be desirable to provide a technique for treating valve insufficiency that reduces the risk of bleeding associated with anticoagulation requirements of cardiopulmonary bypass. In addition, a technique that can be employed on a beating heart would allow the practitioner an opportunity to assess the efficacy of the treatment and potentially address any inadequacies without the need for additional bypass support.

### SUMMARY OF THE INVENTION

To address at least some of these needs, the present invention provides, in exemplary non-limiting embodiments, devices and methods for improving the function of a valve (e.g., mitral valve) by positioning an implantable device outside and adjacent the heart wall such that the device alters the shape of the heart wall acting on the valve. The implantable device may include two anchor ends with a interconnecting member connected therebetween. The anchor ends and the interconnecting member may be positioned on the outside of the heart. Optionally, a protrusion may be connected to the interconnecting member between the anchor ends. The anchor ends may be connected to the heart wall around the dysfunctional valve, and the interconnecting member may be tightened or cinched therebetween. Because the heart wall is generally curved, the act of cinching the interconnecting member between the attached anchor ends may cause the interconnecting member to apply an inward force against the heart wall acting on the dysfunctional valve, and/or may shorten the distance between the anchor ends and thus deform the heart wall inward to act on the dysfunctional valve. The inward force may act on any one of or any combination of valve structures (e.g., valve annulus, papillary muscles, etc.) and/or adjacent anatomical coronary structures. If a protrusion is utilized, it may be used to apply and focus additional force against the heart wall.

In an exemplary aspect of the invention, a device for securing an implant to body tissue may comprise a tissue piercing mechanism configured to rotate relative to the cup such that the tissue piercing mechanism rotates from a first position wherein the tissue piercing mechanism is substantially within the chamber to a second position wherein the tissue piercing mechanism lies substantially over the opening.

According to yet another exemplary aspect, a device for improving heart valve function may comprise an elongate member having a first end and a second end, and an anchoring member associated with each of the first end and the second end and configured to secure the device relative to the heart. Each of the anchoring members may comprise a cup defining a chamber and an opening leading to the chamber and a tissue piercing mechanism configured to rotate relative to the cup such that the tissue piercing mechanism rotates from a first position wherein the tissue piercing mechanism is substantially within the chamber to a second position wherein the tissue piercing mechanism lies substantially over the opening.

In yet a further exemplary aspect, a method for delivering an implant to the heart may comprise providing an implant comprising a substantially elongate member having a first end and a second end, first and second anchoring members associated with each of the first end and the second end and configured to secure the device relative to the heart, and an intermediate portion disposed between the anchoring members along a length of the elongate member. The method may further comprise delivering the elongate member and a first anchoring member attached to the elongate member to the heart, securing the first anchoring member to the heart, advancing the intermediate portion over the elongate member and to the heart, advancing the second anchoring member over the elongate member and to the heart, and securing the second anchoring member to the heart.

In still another exemplary aspect, a delivery system for delivering an implant to a heart may comprise a first catheter configured to simultaneously deliver to the heart an elongate member and a first anchor mechanism attached to a first end of the elongate member. The system also may include a second catheter configured to advance an intermediate component along the elongate member until the intermediate component is adjacent the first anchor mechanism. Additionally, the system may include a third catheter configured to advance a second anchor mechanism along the elongate member to a position adjacent the intermediate component and on a side of the intermediate component opposite the first anchor mechanism.

According to yet another exemplary aspect, a device for improving heart valve function may comprise an elongate member having a first end and a second end and an anchoring member associated with each of the first end and the second end and configured to secure the device relative to the heart such that the device provides a compressive force to an exterior portion of the heart sufficient to alter valve function. The device may further comprise an intermediate component comprising a sleeve configured to be advanced over the elongate member and to be positioned between each anchoring member when the device is implanted in the heart. The sleeve may be configured to distribute the force applied by the elongate member to the heart.

Yet a further exemplary aspect includes a method for delivering an implant to the pericardial space of the heart to treat a heart valve. The method may comprise, from a remote location, inserting a portion of an access device through the pericardium such that the portion automatically is inserted to a predetermined depth beyond the pericardium. After inserting the portion through the pericardium, the method may further include separating the pericardium from the epicardium via the portion, delivering an implant into the pericardial space, and securing the implant relative to the heart such that the implant alters valve function.

According to another exemplary aspect, a system for treating a heart valve may comprise an access device configured to access the pericardial space from a remote location, wherein a portion of the access device is configured to be automatically inserted through the pericardium to a predetermined depth beyond the pericardium and to separate the pericardium from the epicardium. The system may also include an implant configured to be delivered to the pericardial space and to be secured relative to the heart so as to exert a compressive force on the heart sufficient to alter valve function.

### BRIEF DESCRIPTION OF THE DRAWINGS

Aside from the structural and procedural arrangements set forth above, the invention could include a number of other arrangements, such as those explained hereinafter. It is to be understood that both the foregoing description and the following description are exemplary. The accompanying drawings are included to provide a further understanding of the invention and are incorporated in and constitute a part of this specification. The drawings illustrate exemplary embodiments of the invention and, together with the description, serve to explain certain principles. In the drawings,

FIGS. 1A and 1B are bottom and side views, respectively, of an exemplary, non-limiting embodiment of an implantable device utilizing a protrusion;

FIGS. 1C and 1D are bottom and side views, respectively, of an exemplary, non-limiting alternative embodiment of an implantable device without a protrusion;

FIGS. 2A-2C are sectional views of a patient's trunk at the level of the mitral valve of the heart, showing an example of where the implantable devices may be positioned in the short axis view, and showing the effects of the implantable devices on mitral valve function;

FIG. 3 is a sectional view of a patient's heart bisecting the mitral valve, showing an example of where the implantable devices may be positioned in the long axis view;

FIG. 4 is an angiographic illustration of a patient's heart, showing an example of where the implantable devices may be positioned relative to the coronary arteries;

FIGS. 5A-5E are perspective views of more specific embodiments of implantable devices of the present invention;

FIG. 5F is a schematic illustration of a cable locking mechanism for use in any of the implantable devices shown in FIGS. 5A-5E;

FIG. 6A is a perspective plan view of a delivery system for implanting the implantable devices shown in FIGS. 5A-5D;

FIG. 6B is a perspective bottom view of an anchor catheter for use in the delivery system shown in FIG. 6A;

FIG. 7 is a perspective plan view of an alternative delivery system for implanting the implantable devices shown in FIGS. 5A-5D;

FIGS. 8A-8D are perspective plan views of a delivery system for implanting the device shown in FIG. 5E;

FIGS. 9A and 9B are perspective views of a sizing device for use in adjusting the implantable devices shown in FIGS. 5A-5E;

FIG. 10A is a perspective exploded view of an access system to facilitate pericardial access of the delivery systems;

FIG. 10B is a partially sectioned side view of a distal portion of the access device shown in FIG. 10A, illustrating engagement with the pericardial sac;

FIGS. 11A-11D schematically illustrate an alternative access system to facilitate pericardial access of the delivery systems; and

FIG. 12 is an illustration schematically showing an example of one approach for pericardial access.

### DETAILED DESCRIPTION OF EXEMPLARY EMBODIMENTS

The following detailed description should be read with reference to the drawings in which similar elements in different drawings are numbered the same. The drawings, which are not necessarily to scale, depict illustrative embodiments and are not intended to limit the scope of the invention.

The various aspects of the devices and methods described herein generally pertain to devices and methods for treating heart conditions, including, for example, dilatation, valve incompetencies, including mitral valve leakage, and other similar heart failure conditions. Each disclosed device may operate passively in that, once placed on the heart, it does not require an active stimulus, either mechanical, electrical, hydraulic, pneumatic, or otherwise, to function. Implanting one or more of the devices operates to assist in the apposition of heart valve leaflets to improve valve function.

In addition, these devices may either be placed in conjunction with other devices that, or may themselves function to, alter the shape or geometry of the heart, locally and/or globally, and thereby further increase the heart's efficiency. That is, the heart experiences an increased pumping efficiency through an alteration in its shape or geometry and concomitant reduction in stress on the heart walls, and through an improvement in valve function.

However, the devices disclosed herein for improving valve function can be “stand-alone” devices, that is, they do not necessarily have to be used in conjunction with additional devices for changing the shape of a heart chamber or otherwise reducing heart wall stress. It also is contemplated that a device for improving valve function may be placed relative to the heart without altering the shape of the chamber, and only altering the shape of the valve itself. In other words, the devices and methods described herein involve geometric reshaping of portions of the heart and treating valve incompetencies.

The devices and methods described herein offer numerous advantages over the existing treatments for various heart conditions, including valve incompetencies. The devices are relatively easy to manufacture and use, and the transluminal, transthoracic, and surgical techniques and tools for implanting the devices do not require the invasive procedures of current surgical techniques. For instance, these techniques do not require removing portions of the heart tissue, nor do they necessarily require opening the heart chamber or stopping the heart during operation. For these reasons, the techniques for implanting the devices disclosed herein also are less risky to the patient than other techniques. The less invasive nature of these techniques and tools may also allow for earlier intervention in patients with heart failure and/or valve incompetencies.

Although the methods and devices are discussed hereinafter in connection with their use for the mitral valve of the heart, these methods and devices may be used for other valves of the heart for similar purposes. One of ordinary skill in the art would understand that the use of the devices and methods described herein also could be employed for other valves of the heart. The mitral valve has been selected for illustrative purposes because a large number of the disorders occur in connection with the mitral valve.

The devices and methods described herein are discussed herein with reference to the human heart H, but may be equally applied to other animal hearts not specifically mentioned herein. For purposes of discussion and illustration, several anatomical features may be labeled as follows: left ventricle LV; right ventricle RV; left atrium LA; ventricular septum VS; right ventricular free wall RVFW; left ventricular free wall LVFW; atrioventricular groove AVG; mitral valve MV; tricuspid valve TV; aortic valve AV; pulmonary valve PV; papillary muscle PM; chordae tendeneae CT (or simply chordae); anterior leaflet AL; posterior leaflet PL; coaptation line CL; annulus AN; ascending aorta AA; thoracic aorta TA; azygos vein AZV; coronary sinus CS; cardiac vein CV; right coronary artery RCA; left anterior descending artery LAD; obtuse marginal artery OM; circumflex artery CFX; left lung LL; right lung RL; dermal layer DL; sternum ST; xiphoid XPH; diaphragm DPH; and vertebrae VRT.

General Description of Exemplary Implant Devices

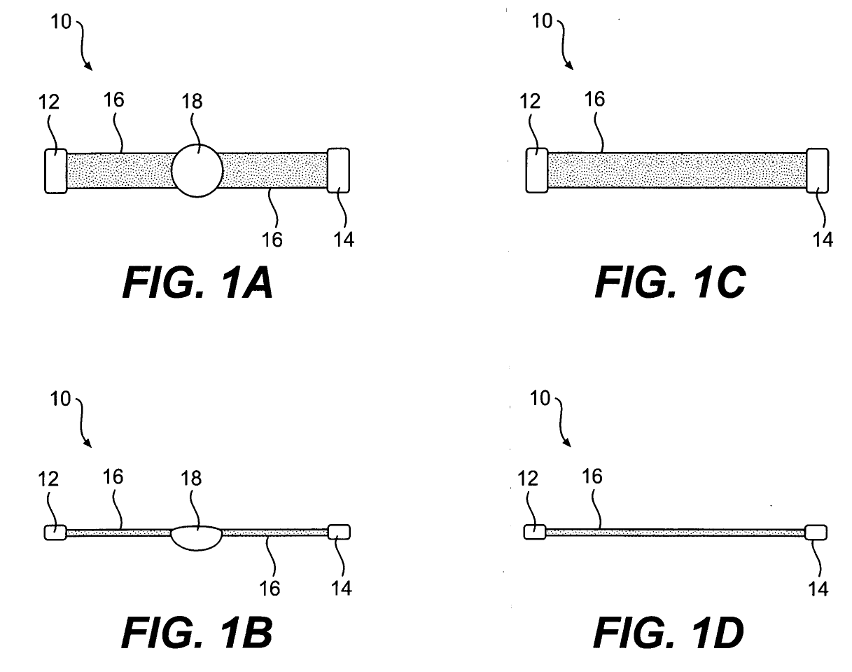

With reference to FIGS. 1A and 1B, a generic implantable device 10 is shown schematically. The implantable device 10 may generally include two or more anchor ends 12/14 with a interconnecting member 16 connected therebetween. The anchor ends 12/14 may be configured to permanently or releasably attach to the outside of the heart wall. The interconnecting member 16 may be selectively tightened or loosened to correspondingly affect the tension between the anchor ends 12/14. A protrusion 18 may be connected to the interconnecting member 16 between the anchor ends 12/14. Alternatively, as shown in FIGS. 1C and 1D, the implantable device 10 may utilize anchor ends 12/14 and interconnecting member 16 alone, without the use of a protrusion 18. With or without protrusion 18, the interconnecting member may be generally flexible to conform to the outer surface of the heart. Protrusion 18 may alternatively be referred to as a space filling member or a focal member. Interconnecting member 16 may alternatively be referred to as an elongate member or as a tension member.

The position of the protrusion 18 may be adjusted relative to the anchor ends 12/14. To accommodate such adjustment, the interconnecting member 16 may be fixedly connected to one or both of the anchor ends 12/14 and adjustably connected to the protrusion 18. Alternatively, the interconnecting member 16 may be fixedly connected to the protrusion 18 and adjustably connected to one or both of the anchor ends 12/14. In both instances, the length of the interconnecting member 16 between the protrusion 18 and the anchor ends 12/14 may be adjusted to change the position of the protrusion 18 relative to the anchor ends 12/14.

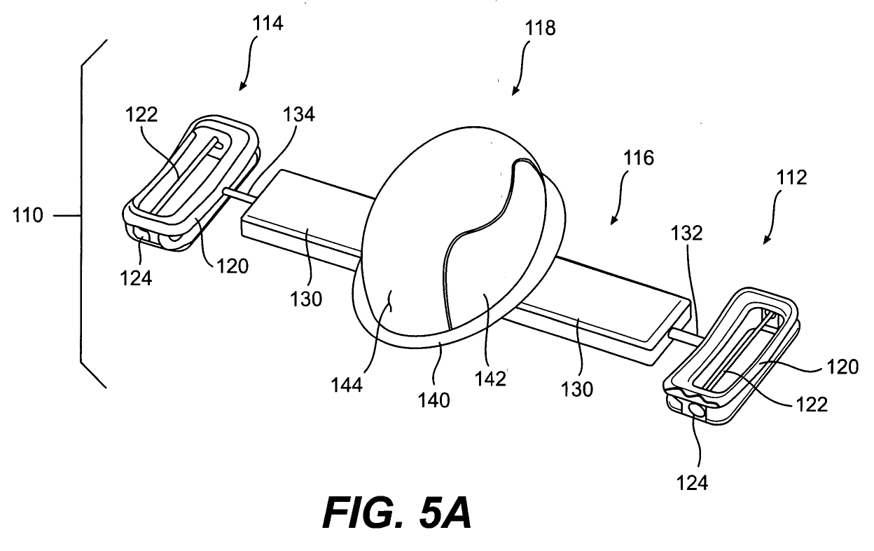

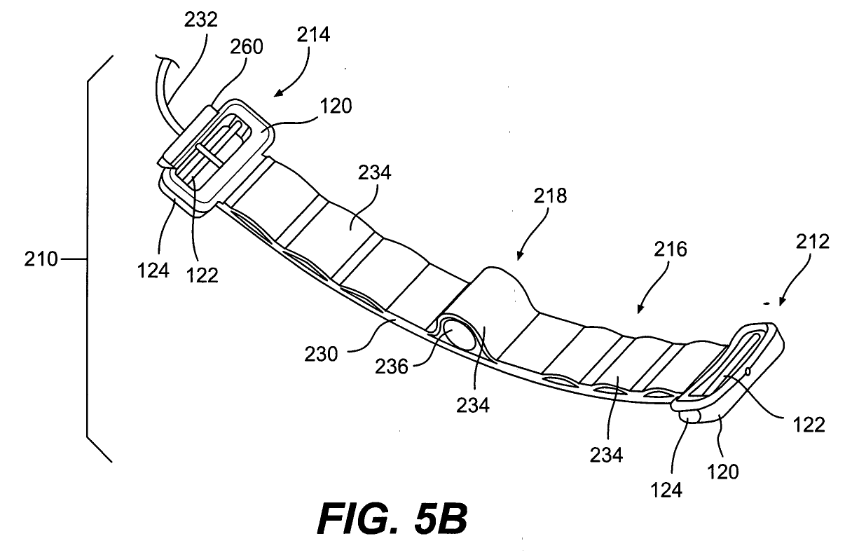

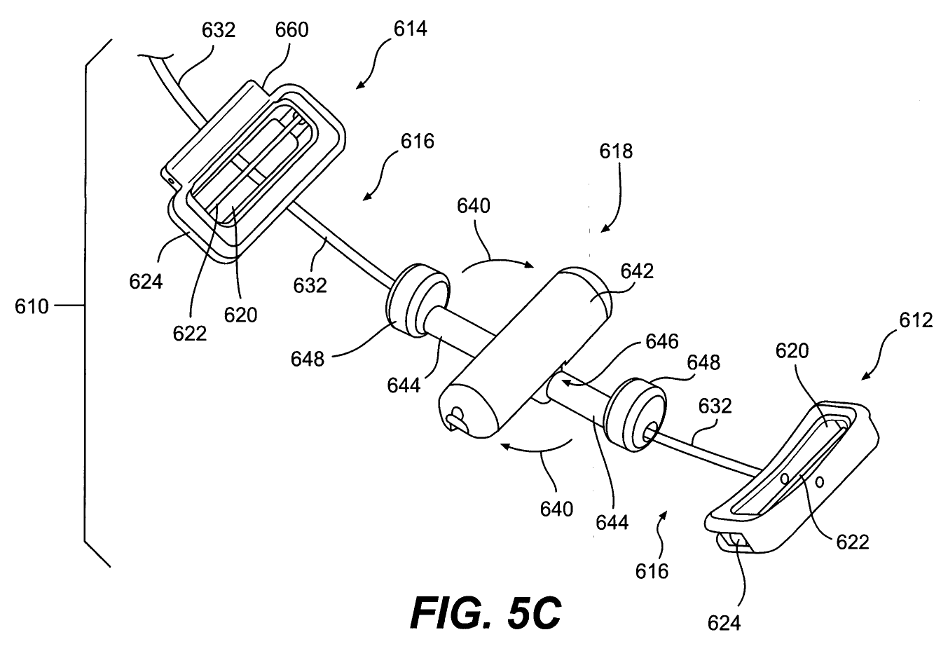

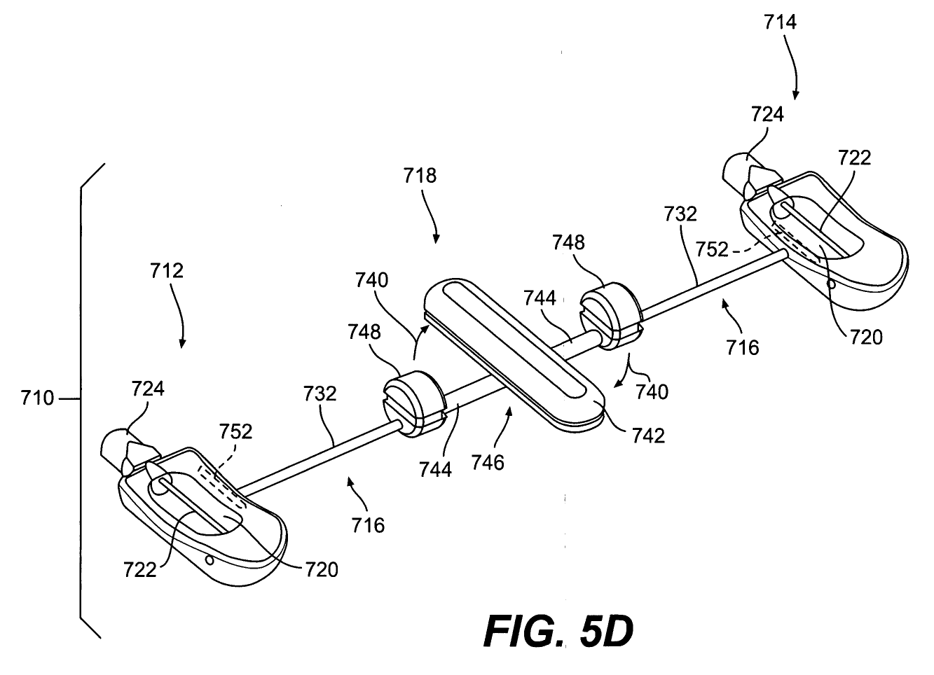

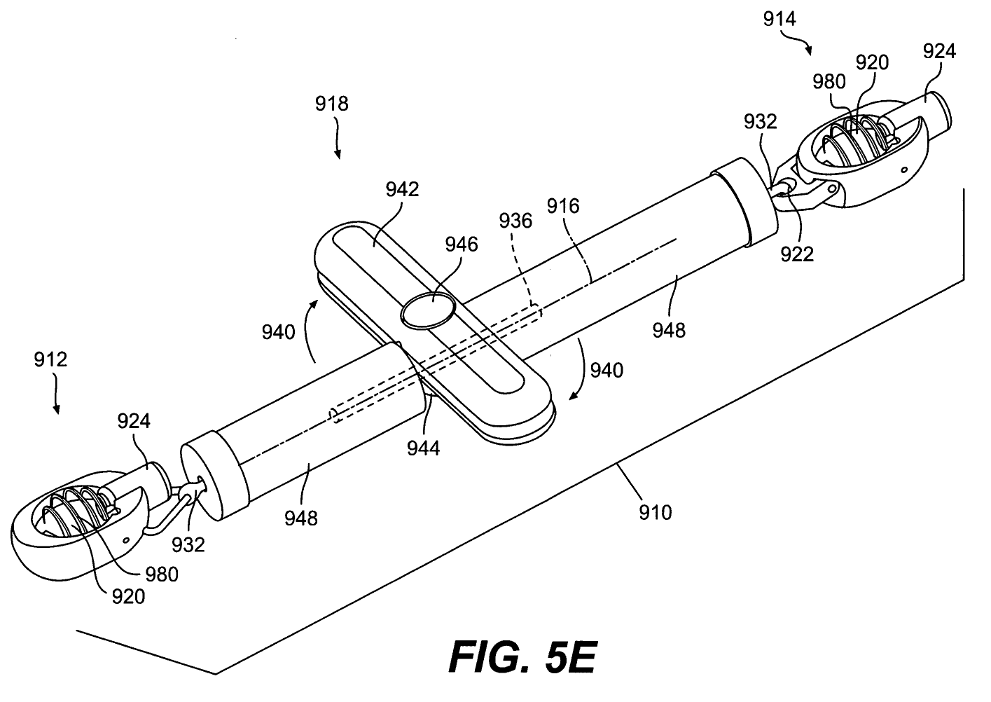

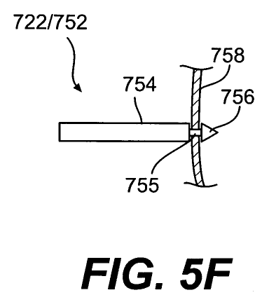

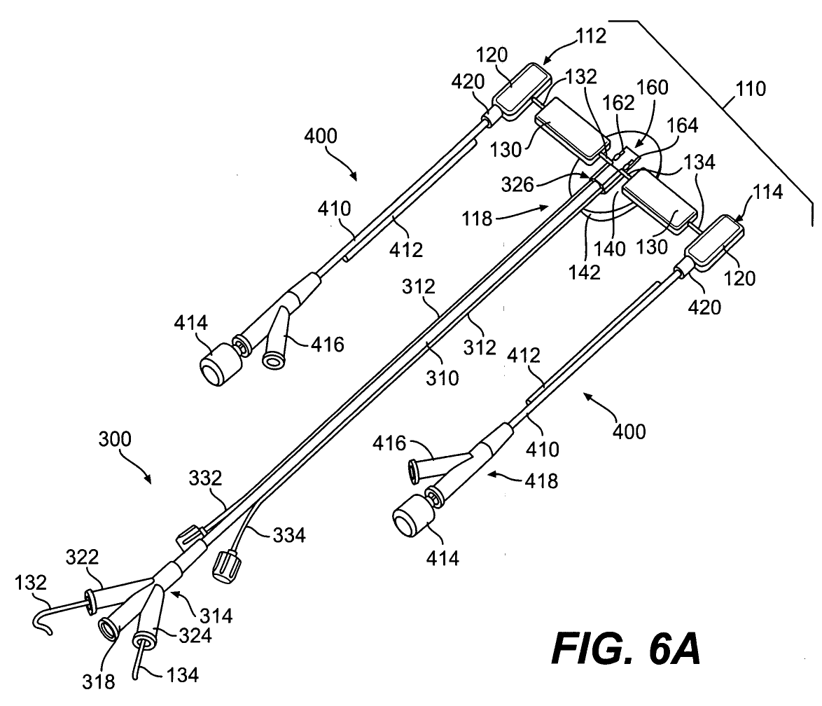

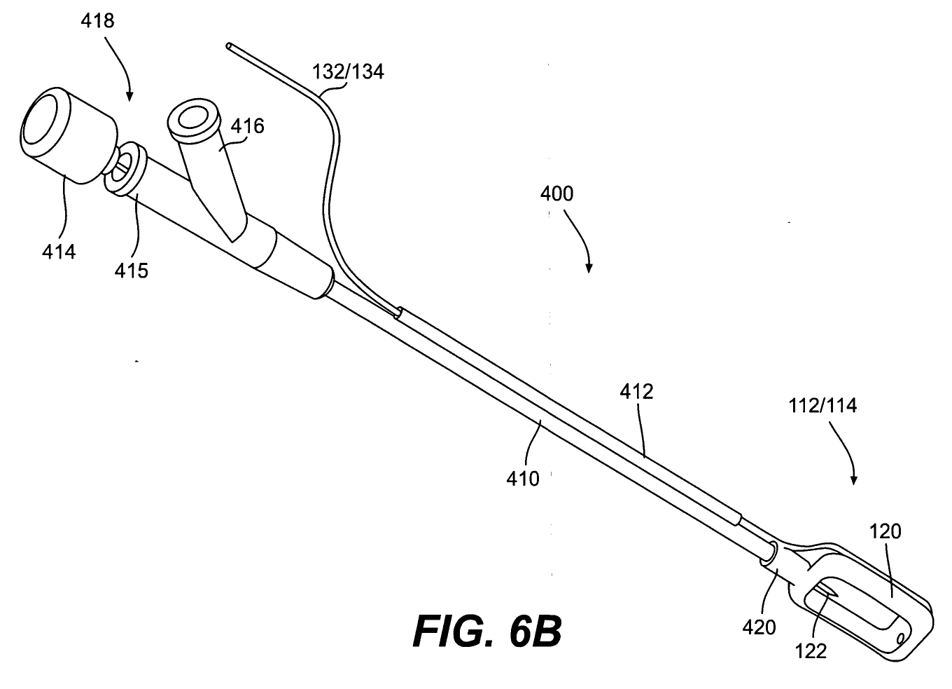

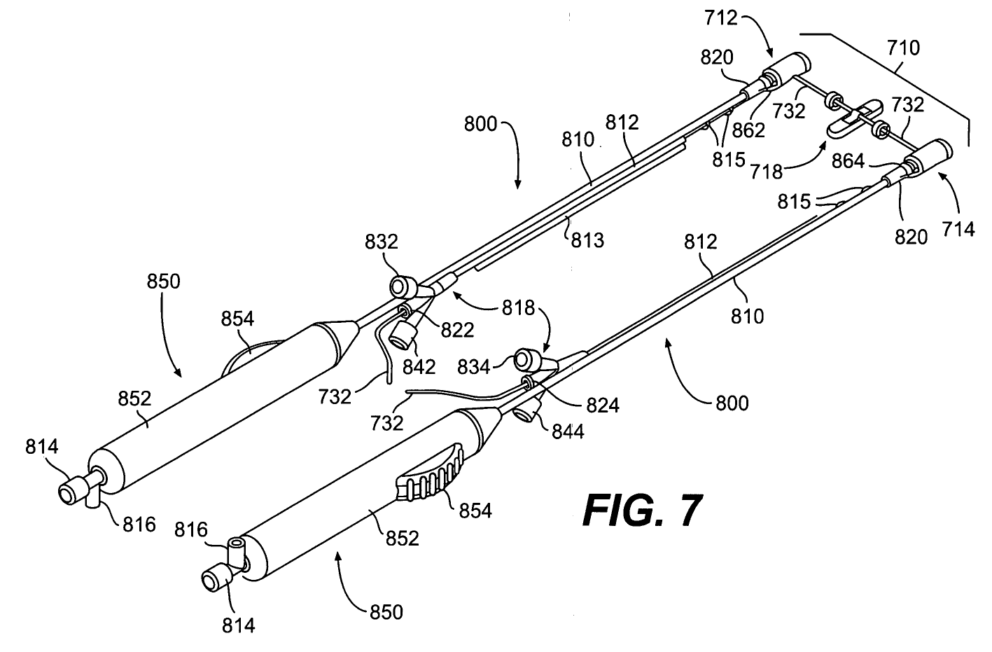

The anchors 12/14 serve to secure the ends of the interconnecting member 16 to the heart wall. The anchors 12/14 may comprise vacuum cups with tissue piercing pins for securement as described in more detail with reference to FIGS. 5A-5E. The anchors 12/14 may be remotely activated as described with reference to FIGS. 6 and 7. The anchors 12/14 may selectively connect to some tissue (e.g., epicardium, myocardium) while remaining free of other tissue (e.g. pericardium). Various alternative anchor embodiments are envisioned, such as tines, screws, sutures, adhesives, etc., and/or a tissue in-growth promoting material (e.g., Dacron fabric). For example, the anchors 12/14 may comprise tines that extend through the epicardium and into the myocardium, and optionally extend through the endocardium into a heart chamber. Additional alternative anchor embodiments are described in U.S. Published Patent Application No. 2004/0148019 to Vidlund et al.

The interconnecting member 16 may be fixed or selectively fixed (i.e., adjustable) to each of the anchors 12/14 and/or the protrusion 18 as described above. The interconnecting member may be made fixed or adjustable using, for example, a lock pin technique as described in more detail with reference to FIGS. 5A-5E.

As an alternative to interconnecting member 16, or in conjunction with interconnecting member 16, pericardial tissue may be used to connect the anchor ends 12/14 and protrusion 18 (if used). For example, a first anchor end 12 may be fixedly secured to both the epicardium and the pericardium using an anchor device with open top and bottom surfaces as described in U.S. Published Patent Application No. 2004/0148019 A1 to Vidlund et al. The second anchor end 14 may be secured to epicardium, and the protrusion 18 may be secured to the pericardium (by using an anchor device for the protrusion 18). The interconnecting member 16 may be fixedly connected to the protrusion 18 and adjustably connected to the second anchor end 14 (or visa-versa) such that the position of the protrusion 18 may be adjusted (e.g., cinched) relative to the second anchor end 14. By virtue of the common pericardial connection between the first anchor 12 and the protrusion 18, cinching the interconnecting member 16 between the protrusion 18 and the second anchor 14 also causes cinching between the protrusion 18 and the first anchor 12, without requiring the interconnecting member 16 to be connected to the first anchor 12.

The interconnecting member 16 may be elongate and will normally be in tension when implanted. The interconnecting member may comprise a flexible and biocompatible multifilament braid in the form of a string or strap, for example. If a string or chord is used, for example, an atraumatic pad (as seen in FIG. 5A) may be disposed on the interconnecting member 16 to avoid stress concentration on the heart wall by the interconnecting member 16.

The interconnecting member 16 may be formed as described in U.S. Pat. No. 6,537,198 to Vidlund et al., the entire disclosure of which is incorporated herein by reference. In particular, the interconnecting member 16 may comprise a composite structure including an inner cable to provide mechanical integrity and an outer covering to provide biocompatibility. The inner cable of interconnecting member 16 may have a multifilament braided-cable of high performance polymeric fibers such as ultra high molecular weight polyethylene available under the trade names Spectra™ and Dyneema™, polyester available under the trade name Dacron™, or liquid crystal polymer available under the trade name Vectran™. The filaments forming the inner cable may be combined, for example, in yarn bundles of approximately 50 individual filaments, with each yarn bundle being approximately 180 denier, and two bundles may be paired together (referred to as 2-ply) and braided with approximately 16 total bundle pairs with approximately 20 to 50 picks per inch (number of linear yarn overlaps per inch). For some applications, it may be desirable to use only one bundle resulting in a cable that is approximately half the size of the example given above.

The outer covering surrounding the inner cable of the interconnecting member 16 may provide properties that facilitate sustained implantation, and may thus be formed of a material that is biocompatible and allows for tissue ingrowth. For example, the outer covering surrounding the inner cable of the interconnecting member 16 may be made of a polyester material (e.g., Dacron) or expanded PTFE (ePTFE). If an atraumatic pad is used, it may be formed of, coated with, or covered by the same or similar material as the outer covering of the interconnecting member to promote tissue in-growth for additional anchoring effect. For example, the atraumatic pad may be formed of ePTFE which is biocompatible, promotes tissue in-growth, and conserves cross-sectional size and shape despite elongation.

The protrusion 18 may comprise a balloon, plug, or other mechanical spacer or structure, and may be fixedly or adjustably connected to the interconnecting member 16. The protrusion 18 may be centered between the anchors 12/14, or may be eccentrically positioned therebetween. One or more protrusions 18 may be used, and the protrusions may have various geometries depending on the desired allocation of forces acting on the heart wall. The protrusion 18 may be coated or covered by a tissue in-growth promoting material to secure the protrusion to the heart wall in the desired position, and the material may be highly elastic or otherwise stretchable to permit expansion of the protrusion 18. Examples of suitable materials include ePTFE and polyester knits.

Description of Exemplary Implant Positions and Functions

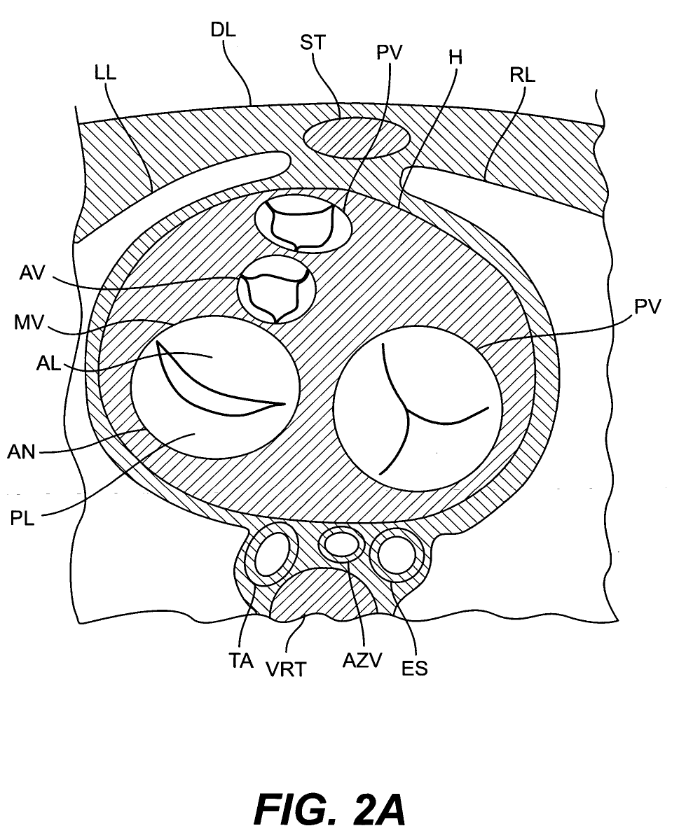

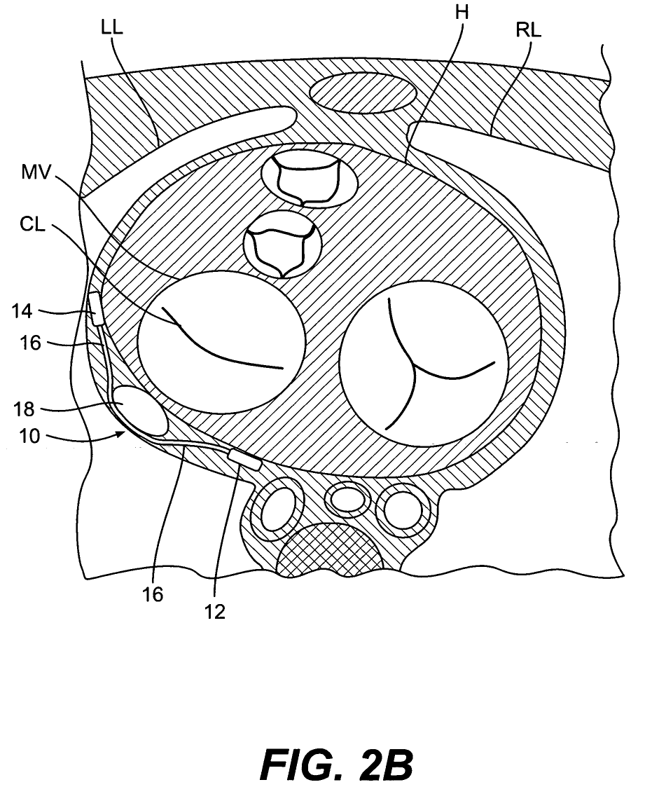

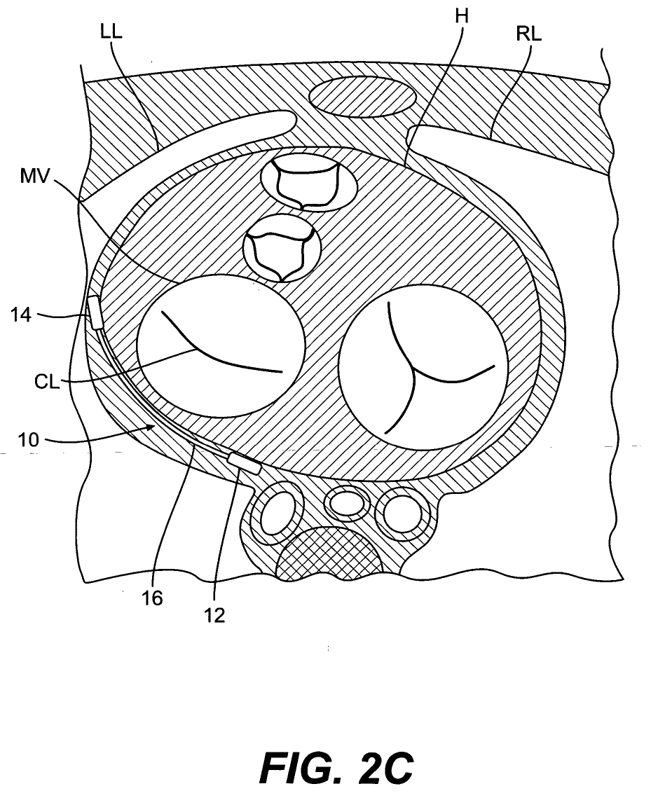

With reference to FIG. 2A-2C, cross sectional views of a patient's trunk at the level of the mitral valve MV of the heart H show the effects of implantable device 10 on mitral valve MV function. As seen in FIG. 2A, an incompetent mitral valve MV is shown during systole, as rendered incompetent by, for example, a dilated valve annulus AN, a displaced papillary muscle PM due to ventricular dilation or other mechanism. With reference to FIGS. 2B and 2C, the implantable device 10 may be positioned outside and adjacent the heart wall such that the device 10 acts on the mitral valve MV. As seen in FIGS. 2B and 2C, the formerly incompetent mitral valve MV is shown during systole as corrected with implantable device 10. The implantable device 10 causes inward displacement of a specific portion of the heart wall adjacent the mitral valve MV resulting in re-configuration and re-shaping of the annulus AN and/or the papillary muscles PM, thus providing more complete closure of the mitral valve leaflets AL/PL during systole, as shown by closed coaptation line CL in FIGS. 2B and 2C.

The implantable device 10 may affect MV function by acting on the adjacent heart wall in several different modes. For example, in one mode of operation, the protrusion 18 (or the interconnecting member 16 if no protrusion is used) of the implantable device 10 may apply or focus an inward force against the heart wall acting on the MV. The back-up force (i.e., the substantially equal and opposite force to the inward force) may be provided by the interconnecting member 16 as fixed to the heart wall by the anchor ends 12/14, the anatomical structure behind the protrusion 18, or a combination thereof. In an alternative mode of operation, the implantable device 10 may act to cinch, compress or otherwise deform the heart wall surrounding the posterior aspect of the mitral valve MV by shortening the circumferential length thereof. In another alternative mode of operation, the implantable device 10 acts to both apply an inward force and cause circumferential shortening. In each of these modes of operation, the inward force and/or circumferential shortening may be applied throughout the cardiac cycle, or may only act during a portion of the cardiac cycle such as during systole.

The implantable device 10 may be implanted in a number of different positions, a select few of which are described herein for purposes of illustration, not necessarily limitation. Generally, the implantable device 10 may be positioned outside the epicardium of the heart wall adjacent the mitral valve MV, such as between the epicardium and pericardium, or between the pericardium and the pleural sac. Also generally, to maximize the effectiveness of the inward force, the implantable device 10 may be positioned to create a normal force against the heart wall that is generally orthogonal to the coaptation line CL formed by the leaflets PUAL. This may be achieved, for example, by positioning the device 10 in a posterior-lateral projection of the mitral valve MV generally orthogonal to the middle tangent of the coaptation line CL as shown in FIGS. 2B and 2C.

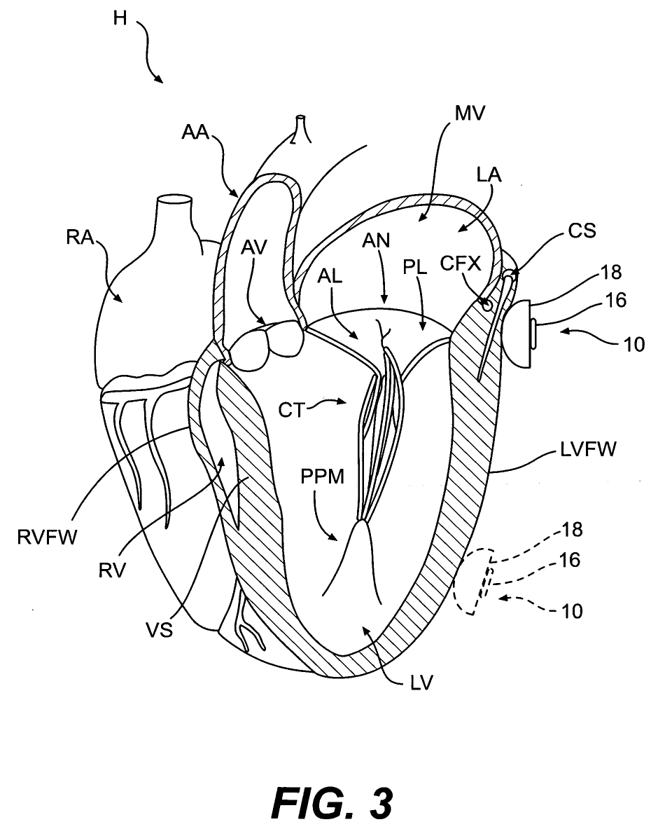

A variety of long axis and short axis positions are contemplated and the particular combination may be selected to have the desired effect. In the short axis view as seen in FIGS. 2B and 2C, the implantable device 10 may extend along all of, a portion of, or beyond the posterior-lateral projection of the mitral valve MV. In the long axis view as seen in FIG. 3, the implantable device 10 may extend along all of, a portion of, or beyond the posterior-lateral projection of the mitral valve MV structures, including the papillary muscles PM, the chordae CT, the leaflets PL/AL, and the annulus AN. For example, the implantable device 10 may be positioned adjacent the annulus AN (e.g., extending slightly above and below the annulus AN near the AV groove), or adjacent the papillary muscles PM (e.g., extending slightly above and below the papillary muscles PM).

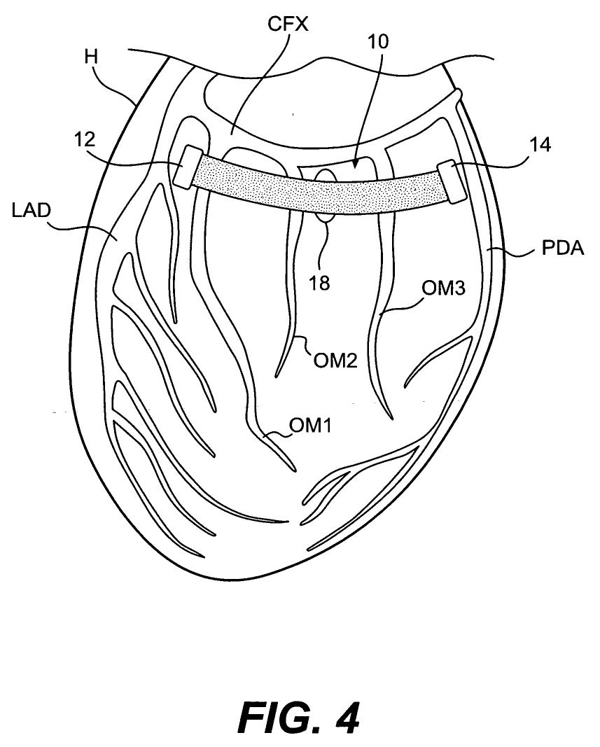

To avoid compression of the coronary arteries which typically reside near the surface of the heart wall, the implantable device 10 may have relatively small contact areas selected and positioned to establish contact with the heart wall while avoiding key anatomical structures. For example, as shown in FIG. 4, the implantable device 10 may be positioned with the first anchor 12 positioned between the proximal left anterior descending artery LAD and the proximal first obtuse marginal OM1, the protrusion positioned inferior of the circumflex artery CFX between the second obtuse marginal OM2 and third obtuse marginal OM3, and the second anchor 14 positioned adjacent the posterior descending artery PDA. Alternatively, the implantable device 10 may have a relatively large surface area in contact with the heart wall to distribute the applied forces and avoid force focal points, particularly on the cardiac vasculature.

Description of Exemplary Delivery Techniques and Approaches

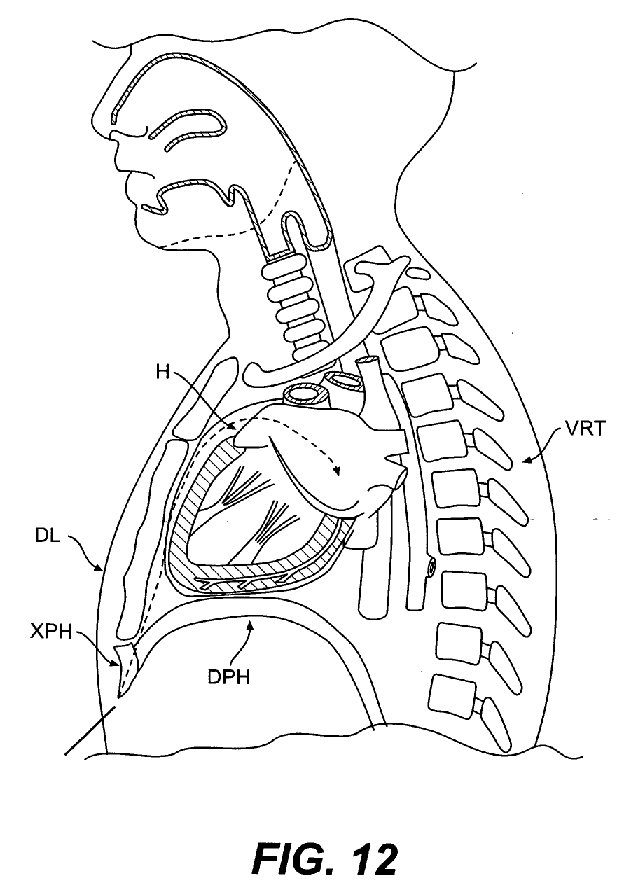

The implantable device 10 may be implanted using one or a combination of various methods and approaches. Generally, these delivery methods may be utilized to implant the device 10 in the pericardial space adjacent the posterior projection of the mitral valve MV. There are a number of different approaches and techniques for positioning the implantable device 10 as such, and these generally include surgical, transluminal and transthoracic techniques. For purposes of illustration, not necessarily limitation, an anterior transthoracic (subxiphoid) approach is described in more detail with reference to FIG. 12. Examples of other suitable approaches are described in more detail in U.S. Published Patent Application No. 2004/0148019 A1 to Vidlund et al.

Exemplary Embodiments of Implant Devices

With reference to FIGS. 5A-5E, perspective views of implantable devices 110, 210, 610, 710, and 910, respectively, are shown. Note that the side of the device 110/210/610/710/910 that faces the heart wall when implanted is the top side in the illustration. Devices 110, 210, 610, and 710 are further exemplary embodiments of the generic embodiment of implantable device 10 described previously, in which similar components have similar nomenclature, and such may be made, used and function in the same or similar manner.

As seen in FIG. 5A, implantable device 110 includes a first anchor 112, a second anchor 114, an interconnecting member 116, and an optional protrusion 118. Each of the first anchor 112, second anchor 114, interconnecting member 116, and protrusion 118 may be loaded with a radiopaque material to render the component visible under x-ray. In this embodiment, the interconnecting member 116 may comprise cables 132 and 134, and the anchors 112 and 114 may comprise vacuum cups 120 with tissue piercing pins 122, as will be described in more detail hereinafter. The anchor members 112 and 114 may be selectively attached, released, and re-attached to the heart, and the protrusion 118 may be selectively adjusted relative to the anchor members 112 and 114 by adjusting the respective lengths of the interconnecting member 116 between each anchor 112, 114 and the protrusion 118. The ends of the interconnecting member 116 may be fixedly attached to the anchors 112 and 114, and adjustment of the length of the interconnecting member 116 is provided by a locking mechanism 160 as seen in and described with reference to FIG. 6A.

The anchors 112 and 114 may comprise a vacuum cup 120 with a tissue piercing pin 122 extending through the interior thereof. The cup 120 may be injection molded, for example, of a suitable biocompatible material such as PEEK, HDPE or PTFE, and the piercing pins 122 may be formed of stainless steel, for example. The piercing pins 122 are slidingly received in two bores disposed in the walls of the cup 120. A locking mechanism such as mating geometry between the bores and the pins may be used to lock the pins in the pierced position as shown. A port 124 in communication with the interior of the cup 120 is provided for releasable connection to an anchor catheter 400 as shown and described with reference to FIGS. 6A and 6B.

Each cup 120 has a rim that conforms to the epicardial surface of the heart wall such that vacuum applied to the cup 120 by the anchor catheter 400 via port 124 draws the epicardial surface of the heart into the interior of the cup. With the epicardial tissue drawn inside the cup by the vacuum, the tissue piercing pin 122 may be advanced to pierce through the heart tissue and lock in the pierced position as shown. A lock mechanism such as illustrated in FIG. 5F may be used to secure pins 122. In this manner, the anchors 112 and 114 may be secured to the outside surface of the heart wall.

The protrusion 118 includes a base 140, an inflatable balloon 142 mounted to the base 140, and an outer covering 144 (shown partially cut-away) extending over the balloon 142. The base 140 may be connected to a locking mechanism 160 (not visible) located on the opposite side of the balloon 142, which in turn is connected to the interconnecting member 116. The base 140 may comprise a flexible or semi rigid polymeric material, and the balloon 142 may comprise a compliant or non-complaint polymeric material conventionally used for implantable balloons. Outer covering 144 may comprise a material that promotes tissue in-growth to provide additional anchoring stability over time. The balloon 142 may be pre-filled, or may be filled during implantation, with a liquid that may solidify (cured) over time. To facilitate inflation of the balloon 142, the interior of the balloon 142 may be in fluid communication with an inflation catheter via a lumen (not visible) extending through the locking mechanism 160 and the base 140 as described with reference to FIG. 6A.

The interconnecting member 116 may comprise two multifilament braided cables 132 and 134. One end of each cable 132 and 134 may be fixedly connected to the anchors 112 and 114, respectively, and the other ends of the cables 132 and 134 may be adjustably connected together by a locking mechanism 160 (not visible) attached to the base 140 of the protrusion 118. The cables 132 and 134 may extend through a pair of atraumatic pads 130 that are secured to the base 140 of the protrusion 118.

As seen in FIG. 5B, implantable device 210 includes a first anchor 212, a second anchor 214, an interconnecting member 216, and a protrusion 218. In this embodiment, the interconnecting member 216 includes a cable 232 extending through a strap 230, with one end of the cable 232 fixedly connected to first anchor 212, and the other end extending through second anchor 214 to which the cable may be selectively locked to adjust the length of the interconnecting member 216. A locking mechanism 260, similar to the locking mechanism 160 discussed with reference to FIG. 6A, may be connected to the second anchor 214 for selective tightening of and fixation to cable 232. Otherwise, anchors 212 and 214 may be the same as anchors 112 and 114 described previously.

Strap 230 may vary in length as a function of the length of the cable 232, and includes a plurality of pockets 234 that may be selectively filled with one or more plugs 236 to serve as the protrusion 218, or the pockets 234 may remain empty. For example, selection of the pockets 234 to fill with plugs 236 may be made to apply an inward force against the heart wall while avoiding or jumping over coronary arteries residing near the surface of the heart wall. Strap 230 may comprise a woven polymeric material such as polyester, and the plug 236 may comprise a solid polymeric material such as PEEK, silicone, HDPE, PTFE, or ePTFE.

As seen in FIG. 5C, implantable device 610 includes a first anchor 612, a second anchor 614, an interconnecting member 616, and a protrusion 618. In this embodiment, the interconnecting member 616 includes cable 632 extending through protrusion 618, with one end of the cable 632 fixedly connected to first anchor 612, and the other end extending through second anchor 614 to which the cable may be selectively locked to adjust the length of the interconnecting member 616. A locking mechanism 660, similar to the locking mechanism 160 discussed with reference to FIGS. 6A and 5F, may be connected to the second anchor 614 for selective tightening of and fixation to cable 632. Anchors 612 and 614 include interior cavities 620 in fluid communication with a vacuum source to accommodate heart tissue for securement thereto by tissue piercing pins 622. A port 624 in communication with the interior of the cup 620 is provided for releasable connection to an anchor catheter 400 or 800 as shown and described with reference to FIGS. 6A/6B and FIG. 7, respectively. Recesses may be provided in each of the anchors 612 and 614 and the protrusion 618 for attachment of tissue in-growth promoting material such as Dacron fabric attached by suture-like material to cover the top, bottom and side surfaces. Otherwise, anchors 612 and 614 may be the same as anchors 112 and 114 described previously.

Protrusion 618 may include a center rotating member 642 coupled to cross member 644 by pivot connection 646. The rotating member 642 may be rotated 90 degrees relative to cross member 644 about pivot 646 as indicated by arrows 640. The rotating member 642 may be rotated as indicated by arrows 640 between a low profile delivery configuration wherein the rotating member 642 is generally aligned with the cross member 644, and a deployed configuration wherein the rotating member 642 is generally orthogonal to the cross member 644 as shown. The rotating member 642 may be rotationally biased to the deployed configuration and may be locked in the deployed configuration. A pair of protrusions 648 may be disposed at opposite ends of the cross member 644. The rotating member 642 in addition to the protrusions 648 may function as protrusions as described previously, while the gap therebetween may be used to avoid critical anatomical structures such as coronary vasculature.

As seen in FIG. 5D, implantable device 710 includes a first anchor 712, a second anchor 714, an interconnecting member 716, and a protrusion 718. In this embodiment, the interconnecting member 716 includes cable 732 fixedly attached to and extending through protrusion 718, with both ends of the cable 732 adjustably connected to the anchors 712 and 714 by pins 752 to selectively lock and adjust the length of the interconnecting member 716. Anchors 712 and 714 include interior cavities 720 in fluid communication with a vacuum source to accommodate heart tissue for securement thereto by tissue piercing pins 722. A port 724 in communication with the interior of the cup 720 is provided for releasable connection to an anchor catheter 400 or 800 as shown and described with reference to FIGS. 6A/6B or FIG. 7, respectively. Recesses may be provided in each of the anchors 712 and 714 and the protrusion 718 for attachment of tissue in-growth promoting material such as Dacron fabric attached by suture-like material to cover the top, bottom (inside anchor) and side surfaces (away from heart surface). Otherwise, anchors 712 and 714 may be the substantially the same as anchors 112 and 114 described previously.

Protrusion 718 may include a center rotating member 742 coupled to cross member 744 by pivot connection 746. The rotating member 742 may be connected to the cross member 744 by an elastic ring and may be rotated 90 degrees relative to cross member 744 about pivot 746 as indicated by arrows 740. The rotating member 742 may be rotated as indicated by arrows 740 between a low profile delivery configuration wherein the rotating member 742 is generally aligned with the cross member 744, and a deployed configuration wherein the rotating member 742 is generally orthogonal to the cross member 744 as shown. The rotating member 742 may be rotationally biased to the deployed configuration and may be locked in the deployed configuration. A pair of protrusions 748 may be disposed at opposite ends of the cross member 744. The rotating member 742 in addition to the protrusions 748 may function as protrusions as described previously, while the gap therebetween may be used to avoid critical anatomical structures such as coronary vasculature.

As seen in FIG. 5E, implantable device 910 includes a first anchor (or posterior cup) 912, a second anchor (or anterior cup) 914, an interconnecting member 916, and a protrusion 918. In this embodiment, the interconnecting member 916 comprises a cable 932 extending through protrusion 918, with one end of the cable 932 fixedly attached (e.g., tied) to first anchor 912 and the other end adjustably connected (e.g., by pin shown in FIG. 5F) to second anchor 914 to selectively lock and adjust the length of the interconnecting member 916 between the anchors 912, 914. The second anchor 914 includes a guide 922 through which the interconnecting member 932 may extend.

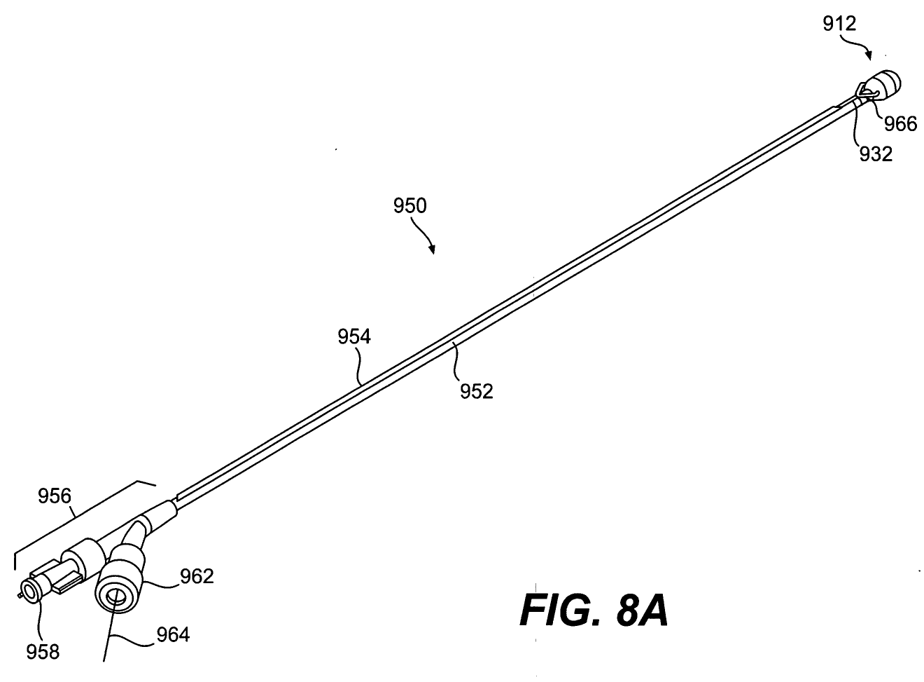

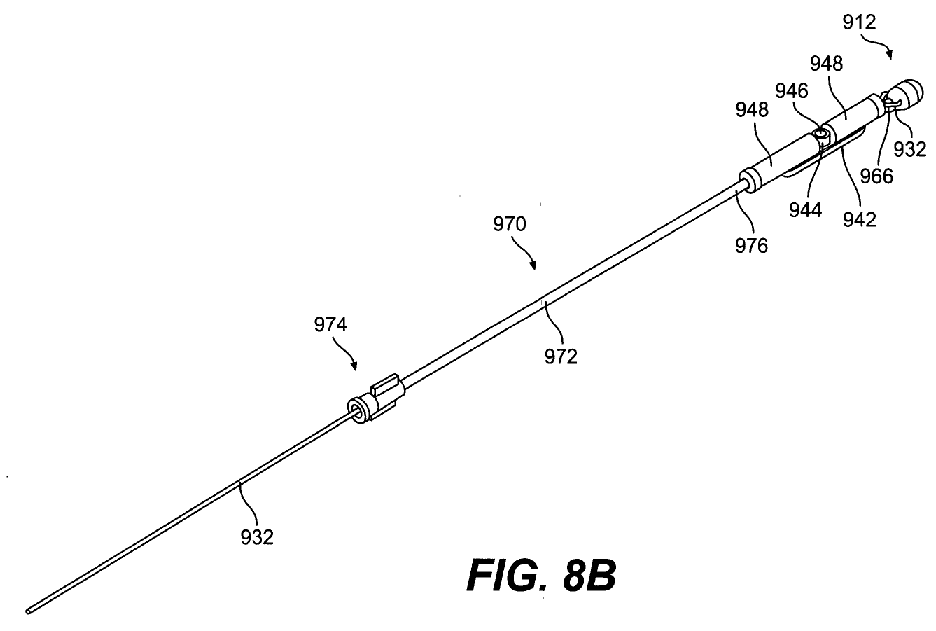

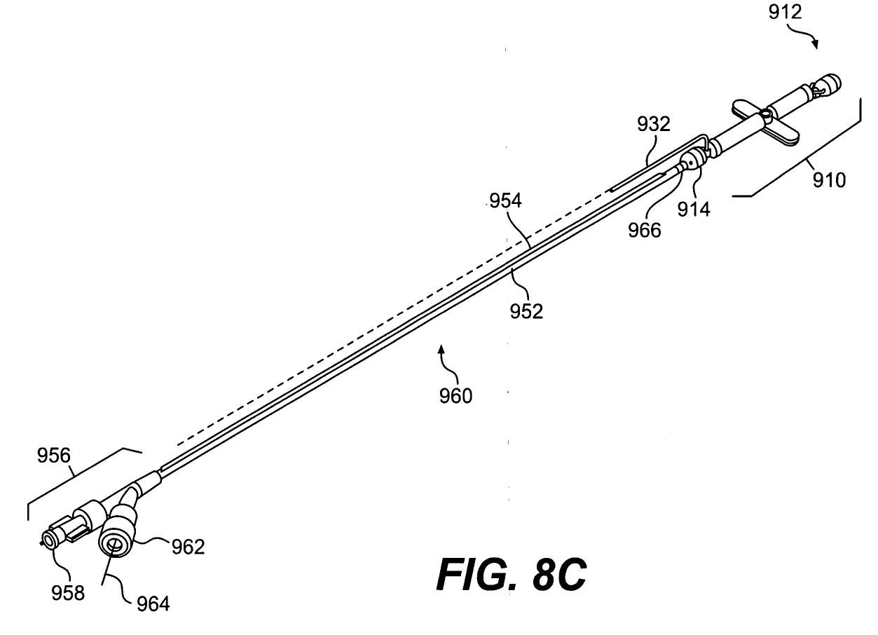

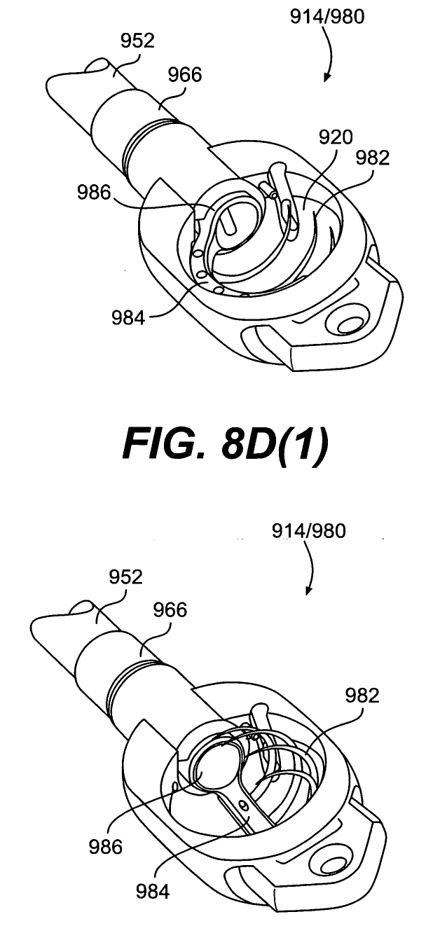

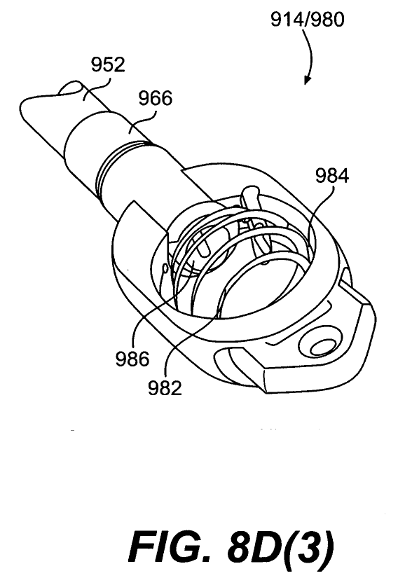

Anchors 912 and 914 include interior cavities 920 in fluid communication with a vacuum source to accommodate heart tissue for securement thereto by bail mechanisms 980. Bail mechanisms 980 are shown in the closed position (i.e., with tissue piercing pins extending through tissue), and are described in more detail with reference to FIGS. 8D(1)-8D(3). A port connector 924 in communication with the interior of the cup 920 is provided for releasable connection to an anchor catheter 950, 960 as shown and described with reference to FIGS. 8A and 8C. Recesses may be provided in each of the anchors 912 and 914 and the protrusion 918 for attachment of tissue in-growth promoting material such as Dacron fabric attached by suture-like material to cover the top, bottom (inside anchor) and side surfaces (away from heart surface). Otherwise, anchors 912 and 914 may be the substantially the same as anchors 112 and 114 described previously.

Protrusion 918 may include a center rotating member 942 which rotates about base 944 and pivot 946. Interconnecting member 916 extends through base 944 and may be slidable relative thereto. The rotating member 942 may be connected to the base 944 and/or pivot 946 by an elastic ring or other biasing member, and may be rotated 90 degrees relative to base 944 and interconnecting member 916 about pivot 946 as indicated by arrows 940. The rotating member 942 may be rotated as indicated by arrows 940 between a low profile delivery configuration wherein the rotating member 942 is generally aligned with and parallel to the interconnecting member 916, and a deployed configuration wherein the rotating member 942 is generally orthogonal to the interconnecting member 916 as shown. The rotating member 942 may be rotationally biased to the deployed configuration and may be locked in the deployed configuration.

A rigid member 936 such as a hypotube may be disposed about the interconnecting member 916 to assist in the transfer and distribution of force from the interconnecting member 916 to the protrusion 918. A pair of sleeves 948 may be disposed on either side of rotating member 942 about the rigid member 936 and the interconnecting member 916. The sleeves 948 may be longitudinally compliant to accommodate changes in length of the interconnecting member 916 between the anchors 912, 914. The sleeves 948 may be radially compliant to accommodate anatomical contours and avoid stress concentration points. The rotating member 942 may function as a protrusion as described previously, while the rigid member 936 and sleeves 948 may function to more broadly distribute the load applied by the interconnecting member 916. To this end, sleeves 948 may be formed of a conformable material such as expanded PTFE (ePTFE) to avoid compromising critical anatomical structures such as coronary vasculature.

As seen in FIG. 5F, an example of a lock mechanism is shown to secure tissue piercing pins 722 and/or cable piercing pins 752. The pins 722/752 may include a cylindrical shaft 754 and a sharpened tip 756 with a recess 755 therebetween. A braided multifilament material 758 such as Spectra™ is provided distal of the pins 722/752 in the anchor housing 712/714 to catch the recess 755 of the pins 722/752 when the tip 756 is advanced therethrough. This effectively locks the pins 722/752 in the advanced position to secure the interconnecting member 716 to the anchors 712 and 714 and/or to secure the anchors 712 and 714 to the heart tissue as will be described in more detail hereinafter.

Exemplary Embodiments of Delivery Devices

With reference to FIG. 6A, an example of a delivery system for delivery and implanting device 110 is shown. The delivery system generally includes a delivery catheter 300 and two anchor catheters 400, all of which are releasably connected to the implantable device 110. The illustrated delivery system is particularly suitable for delivering implantable device 110, but may also be modified for delivery of implantable devices 210, 610 and 710. The delivery system may be configured in terms of size, length, flexibility, radiopacity, etc., to facilitate a transthoracic delivery approach such as the subxiphoid delivery approach described with reference to FIG. 12.

The delivery catheter 300 includes a tubular shaft 310 defining an inflation lumen and two cable lumens extending therethrough. A pair of push tubes 312 extend along side the tubular shaft 310 and slidably accommodate push rods 332 and 334. The distal ends of the tubular shaft 310 and push tubes 312 are coupled to the locking mechanism 160 by a release mechanism 326 such as a threaded, pinned or other releasable connection, such as the pin mechanism illustrated in FIG. 5F. The push rods 332 and 334 may be advanced or retracted to selectively actuate individual pins 162 and 164 respectively in the lock mechanism 160 such that the pins 162 and 164 pass through the cables 132 and 134, respectively, and thus lock the cables relative thereto. Reference may be made to published U.S. Patent Application No. 2003/0050529 to Vidlund et al., the disclosure of which is incorporated herein by reference, for an example of a similar locking mechanism.

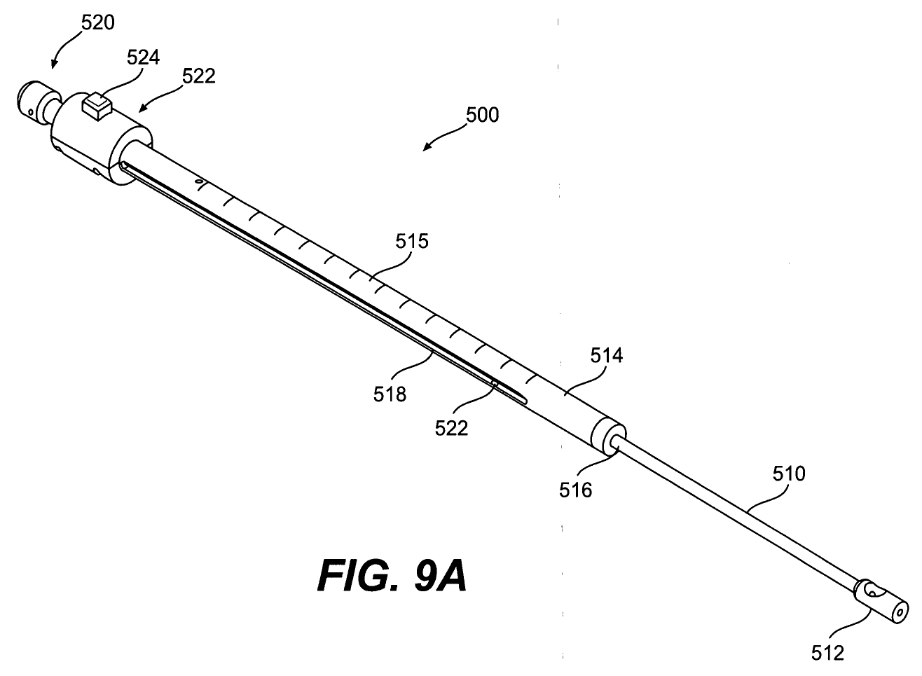

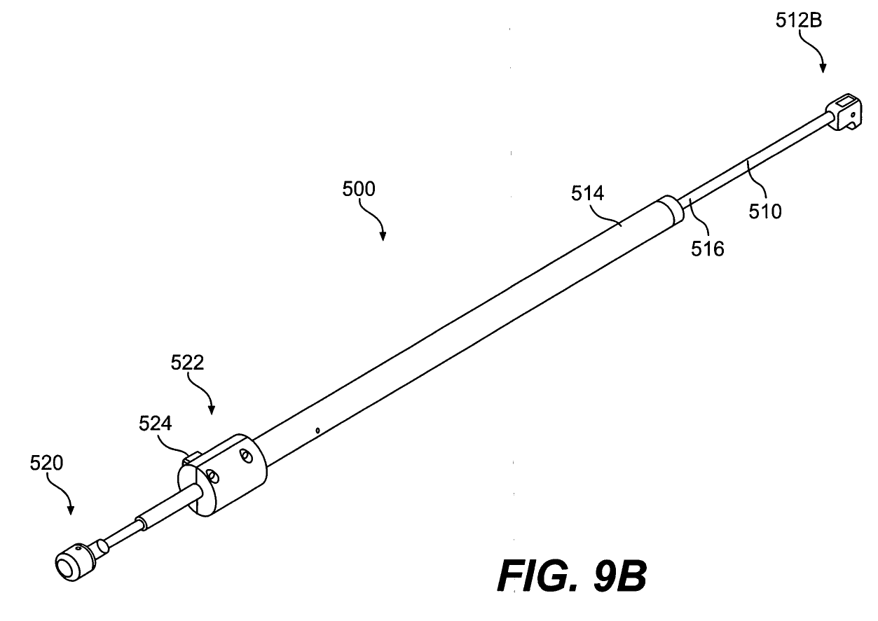

The proximal end of the tubular shaft 310 is connected to a manifold including connectors 322 and 324 and inflation port 318. The inflation lumen of the tubular shaft 310 provides fluid communication between the interior of the balloon 142 and the inflation port 318 of the manifold 314 for connection to an inflation device (not shown) to facilitate inflation and deflation of the balloon 142. If no balloon 142 is used, the inflation lumen and associated parts may be eliminated. The cable lumens of the tubular shaft 310 accommodate the proximal portions of the cables 132 and 134 for connection to a sizing device 500 via connectors 322 and 324 as described with reference to FIGS. 9A and 9B, and for positioning the implant 110 relative to the anchors 112 and 114.

With additional reference to FIG. 6B, the anchor catheters 400 are essentially mirror constructions of each other, and include a tubular shaft 410. A slit guide tube 412 extends alongside a portion of the tubular shaft 410 to guide the cable 132/134 before the delivery catheter 300 is advanced as will be discussed in more detail hereinafter. A proximal end of the tubular shaft 410 is connected to a manifold 418 including a vacuum port 416 and a gasketed port 415 containing a push rod 414. A distal end of the tubular shaft 410 is releasably connected to the anchor 112/114 by a release mechanism 420 that may comprise a threaded, pinned or other releasable connection, for example. The tubular shaft 410 includes a vacuum lumen (not visible) extending therethrough to provide a fluid path from the interior of the cup 120 to the vacuum port 416 to facilitate connection to a vacuum source. The push rod 414 is disposed in the vacuum lumen of the catheter shaft 410 and may be slid therethrough to selectively advance or retract the piercing pin 122 in the cup 120.

With reference to FIG. 7, an example of a delivery system for delivery and implanting device 710 is shown. The delivery system generally includes two anchor catheters 800, both of which are releasably connected to the implantable device 710. The illustrated delivery system is particularly suitable for delivering implantable devices 210, 610 and 710, but may also be modified for delivery of implantable device 110. The delivery system may be configured in terms of size, length, flexibility, radiopacity, etc., to facilitate a transthoracic delivery approach such as the subxiphoid delivery approach described with reference to FIG. 12.

The anchor catheters 800 are essentially mirror constructions of each other (with the exception of split tube 813), and include a tubular shaft 810 comprising a directional catheter construction connected to a handle 850. The directional catheter shaft 810 and associated handle 850 are available from Medamicus, Inc. of Plymouth, Minn. Handle 850 generally includes a grip portion 852 and a thumb knob 854 which actuates control wires in the directional catheter shaft 810 to permit selective bidirectional lateral deflection of the distal end thereof. The directional catheter shaft 810 and associated handle 850 accommodate a push rod (not visible) extending therethrough for actuation of the tissue piercing pin 722. The push rod for the tissue piercing pin 722 may comprise a stainless steel mandrel, for example, with a distal end abutting the proximal end of the tissue piercing pin 722, and a proximal end connected to a knob 814. The directional catheter shaft 810 and associated handle 850 also accommodate a vacuum lumen (not visible) extending therethrough to define a fluid path to the interior 720 of the anchor 712/714, such that a vacuum source (not shown) may be connected to vacuum port 816 on the handle 850 to provide suction at the anchor 712/714 to facilitate stabilization and securement to the outside of the heart wall.

Each of the anchor catheters 800 also includes a side tube 812 coextending with the directional catheter shaft 810. Side tube 812 accommodates the interconnecting member 732, a push rod (not visible) for actuation of the interconnecting member piercing pin 752, and a pull wire (not visible) for release of the anchor 712/714 as described in more detail below. The interconnecting member 732 extends through the side tube 812 from a proximal port 822/824 through the anchor 712/714 to the protrusion 718. To accommodate the interconnecting member 732 during initial delivery of the implant 710, a slotted side tube 813 may be provided on one of the catheters 800.

The push rod for the interconnecting member piercing pin 752 may comprise a stainless steel mandrel, for example, with a distal end abutting the proximal end of the interconnecting member piercing pin 752, and a proximal end connected to knob 832/834. A pair of guide loops 815 may be provided distal of the side tube to guide the interconnecting member 732, and a guide tube 862/864 may be provided distal of the side tube 812 to guide the push rod for the interconnecting member piercing pin 752.

The distal end of the directional catheter shaft 810 is connected to anchor 712/714 by a releasable connection 820, which may comprise a threaded type connection or a cotter pin type connection, for example. In the illustrated embodiment, the releasable connection 820 comprises a cotter pin type connection, with the pull wire (not visible) proximally connected to pull knob 842/844, and distally extending through aligned holes (not visible) in the anchor 712/714 and in the fitting on the distal end of the directional catheter shaft 810. By pulling proximally on pull knob 842/844, the anchor 712/714 may be released from the distal end of the directional catheter shaft 810.

With reference to FIGS. 8A-8D, an example of a delivery system for delivery and implanting device 910 is shown. The illustrated delivery system is particularly suitable for delivering implantable device 910, but may also be modified for delivery of other implantable devices described herein. The delivery system may be configured in terms of size, length, flexibility, radiopacity, etc., to facilitate a transthoracic delivery approach such as the subxiphoid delivery approach described with reference to FIG. 12.

The illustrated delivery system generally includes two anchor delivery catheters 950 (as seen in FIG. 8A) and 960 (as seen in FIG. 8C) for the delivery of the anchors 912 and 914, respectively, of the implantable device 910. The delivery system also includes a delivery catheter 970 (as seen in FIG. 8B) for the delivery of the intermediate components (942, 944, 946, 948) disposed between the anchors 912 and 914 of the implantable device 910.

With specific reference to FIGS. 8A and 8C, the anchor catheters 950 and 960 are essentially mirror constructions of each other, and each includes a tubular shaft 952 defining a suction lumen extending therein. The proximal end of the tubular shaft 952 may be connected to a manifold 956 and the distal end of the shaft 952 may include releasable connectors 966 for releasable connection to the anchors 912 and 914 of the implantable device 910. The releasable connectors 966 may comprise threaded connections or pinned connections, for example. Optionally, the tubular shaft 952 may comprise a directional catheter construction as described elsewhere herein. Each catheter 950 and 960 may include a side tube 954 extending alongside the tubular shaft 952 for containment of the interconnecting member (e.g., cable) 932. Optionally, the side tube 954 may be slitted or slotted for easy removal of the cable 932.

The manifold 956 may include a winged suction port 958 in communication with the suction lumen extending through the tubular shaft 952 to the cavity 920 in the anchor 912, 914. Connection of the suction port 958 to a vacuum source applies suction to the anchor 912, 914 for temporary stabilization and securement to the outside of the heart wall. The manifold 956 may also include a side port 962 through which an actuation member (e.g., pull wire) 964 may extend for actuation of the release mechanism 966. For example, pulling the pull wire 964 may pull a pin in the release mechanism 966 to disconnect the distal end of the shaft 952 from the anchor 912, 914. The winged port 958 may be connected to a torque cable (not visible) extending through the tubular shaft 952 to the bail mechanism 980, such that rotation of the winged port 958 causes rotation of the torsion cable and actuation of the bail mechanism 980 as shown and described in more detail with reference to FIGS. 8D(1)-8D(3).

With specific reference to FIG. 8B, delivery catheter 970 includes a tubular shaft 972 having a lumen extending therethrough to slidably accommodate the interconnecting member (cable) 932. The proximal end of the tubular shaft 972 may be connected to a hub 974, and the distal end 976 of the tubular shaft 972 may abut the intermediate components (942, 944, 946, 948) of the implantable device 910. The delivery catheter 970 may be used to advance (i.e., push) the intermediate components (942, 944, 946, 948) along the interconnecting member (cable) 932 once the first anchor 912 has been deployed.

With reference to FIG. 8C, the second anchor catheter 960 is essentially a mirror construction of the first anchor catheter 950 described previously. Using the second anchor catheter 960, the second (anterior) anchor 914 may be advanced along the interconnecting member 916 toward the mid components as will be described in more detail hereinafter.

With reference to FIGS. 8D(1)-8D(3), the operation of the bail mechanism 980 is shown in succession from the retracted position as shown in FIG. 8D(1), to the intermediate position as shown in FIG. 8D(2), to the fully deployed (anchored) position as shown in FIG. 8D(3). Although only the second anchor 914 is illustrated in FIGS. 8D(1)-8D(3), the bail mechanism 980 is common to both first anchor 912 and second anchor 914.

Bail mechanism 980 may be disposed in the cavity 920 of each anchor 912, 914. Alternatively, the bail mechanism 980 may be disposed around the exterior of the cup of each anchor 912, 914 to provide a more effective seal between the rim of the cup and the tissue when vacuum is applied, and to avoid interfering with tissue sucked into the cup. The bail mechanism 980 may include a plurality of curved tissue piercing pins 982 extending from swing arm 984. Swing arm 984 is connected to pivot base 986 which is connected to torque cable (not visible) extending through shaft 952. Rotation of the torque cable causes rotation of the pivot base 986 which causes the swing arm 984 to sweep across the bottom of the cavity 920 and the tissue piercing pins 982 to rotate out of the cavity 920 and into heart tissue. Stop pins may be disposed in the cavity 920 to limit rotation and/or lock the bail in the fully deployed position.

With reference to FIGS. 9A and 9B, a sizing device 500 is shown for adjusting the tension of interconnecting member 116, 216, 616, 716 or 916 and in particular cable members 132/134, 232, 632, 732 or 932. Sizing device 500 includes an elongate interconnecting member receiving tube 510 having a distal end including an engagement member 512 for releasable connection to the implant device 110, 210, 610, 710, 910. FIG. 9A shows a straight engagement member 512A suitable for devices 110, 210, 610 and 710, and FIG. 9B show an angled engagement member 512B suitable for device 910. Elongate interconnecting member receiving tube 510 includes a proximal end 516 connected to a preferably clear measuring tube 514 having a measuring scale 515 marked thereon. An inner tube 518 is disposed in the measuring tube 514 and is connected to a proximal end of the cable member to be tensioned. A lock mechanism 522 and release button 524 (biased in locked position) are connected to the proximal end of the measuring tube 514 to selectively lock the inner tube 518 relative to the measuring tube. A pin 522 protruding from inner tube 518 extends through a slot in measuring tube 514 to prevent relative rotation. An indicator (not visible) on the inner tube 518 adjacent the pin 522 is visible through transparent measuring tube 514 to facilitate linear measurement relative to scale 515.

To connect the cable to the inner rod or tube 518, the cable 132/134, 232, 632, 732 or 932 is threaded through receiving tube 510, through measuring tube 514, through the inner tube 518, and placed in a retaining mechanism 520 disposed on the inner tube 518. Engagement member 512 may be connected to one of the connectors 322/324 or 822/824 on the delivery catheter, directly to the lock mechanism 160 of device 110, directly to the lock mechanism of device 210, or to the anterior cup 914 of device 910. With this arrangement, the inner tube 518 may be pulled proximally relative to the measuring tube 514 to apply tension to the cable and thus selectively adjust the tightness or degree of cinching of the implantable device 110/210/610/710/910, and/or selectively adjust the position of the protrusion relative to the anchor ends.

Exemplary Embodiments of Access Devices

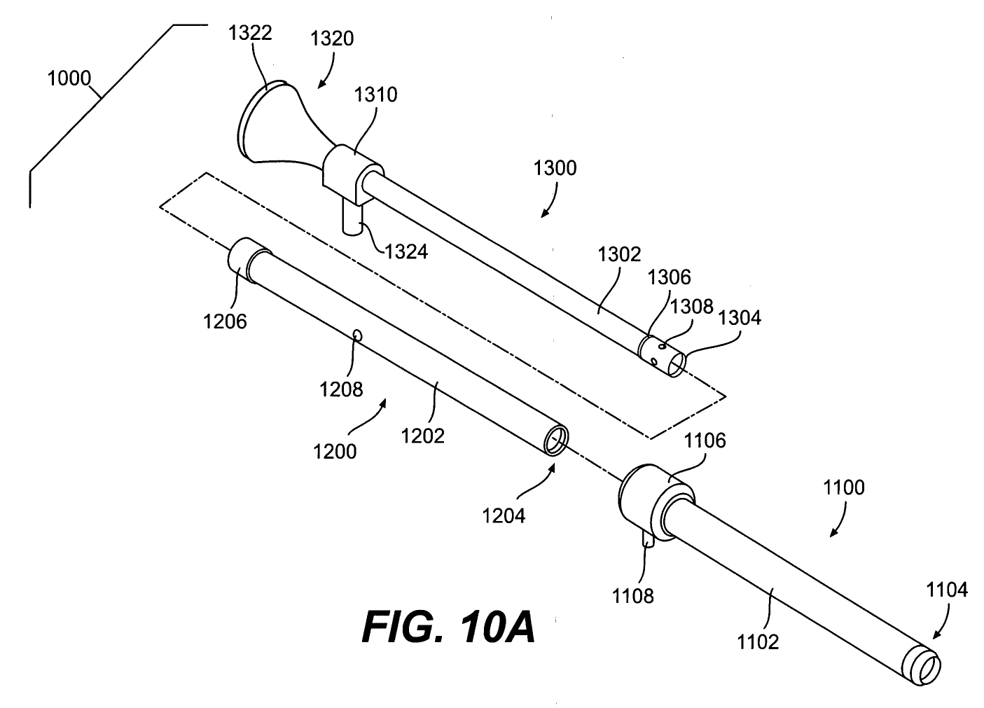

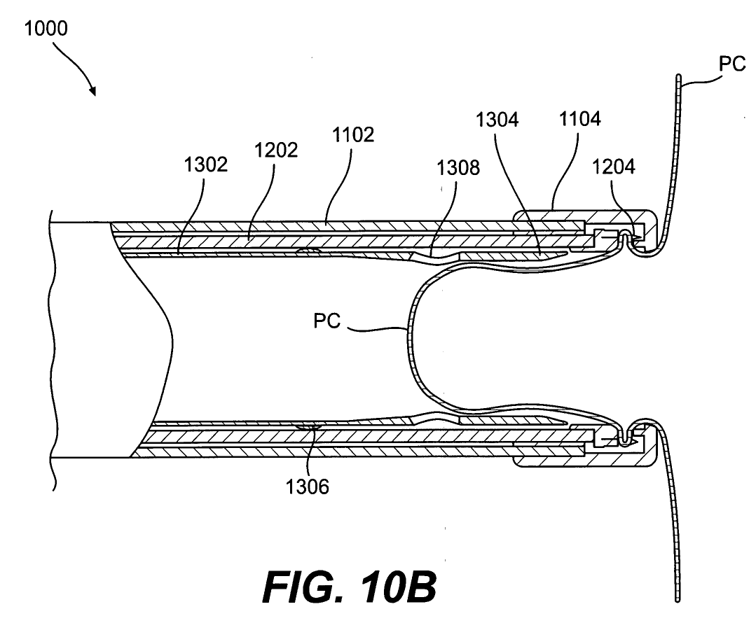

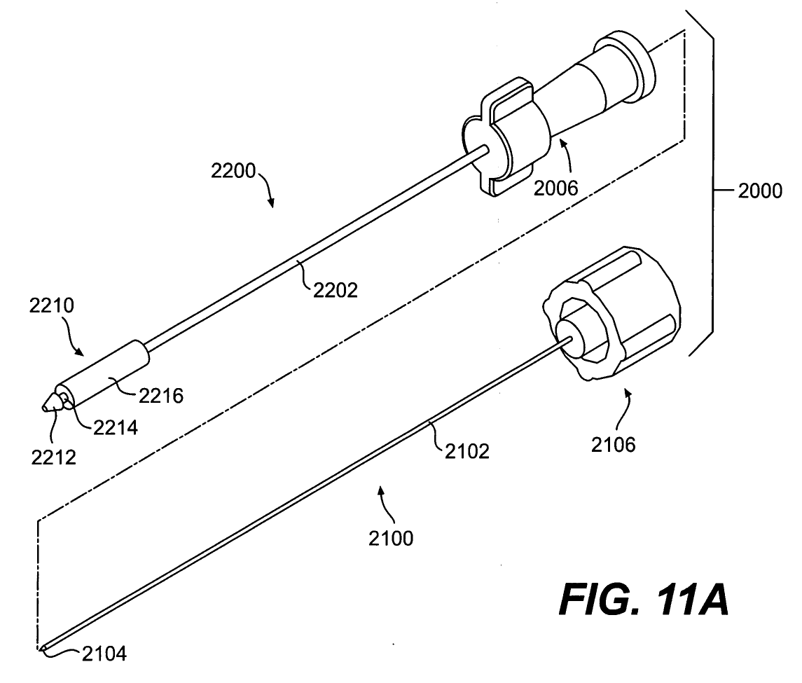

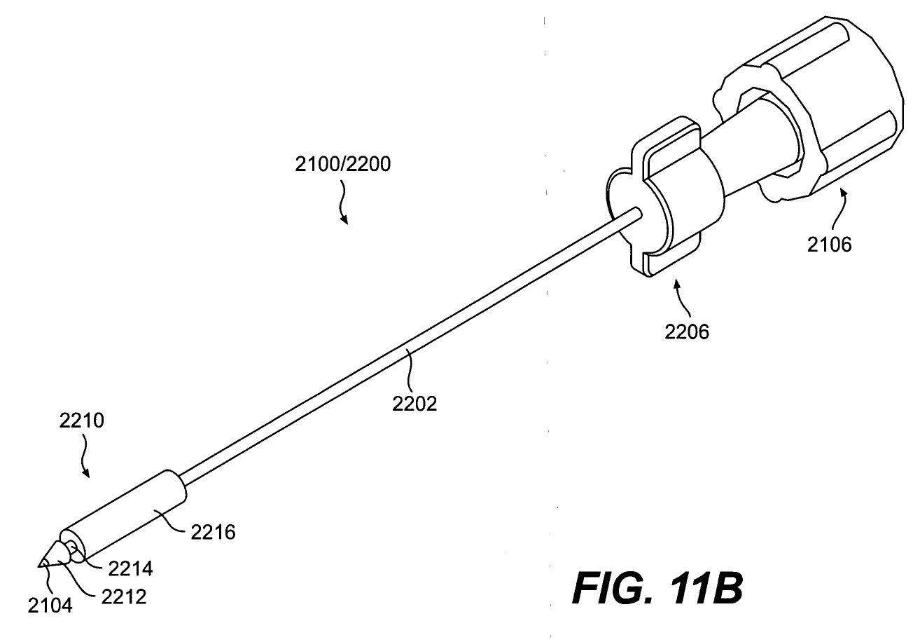

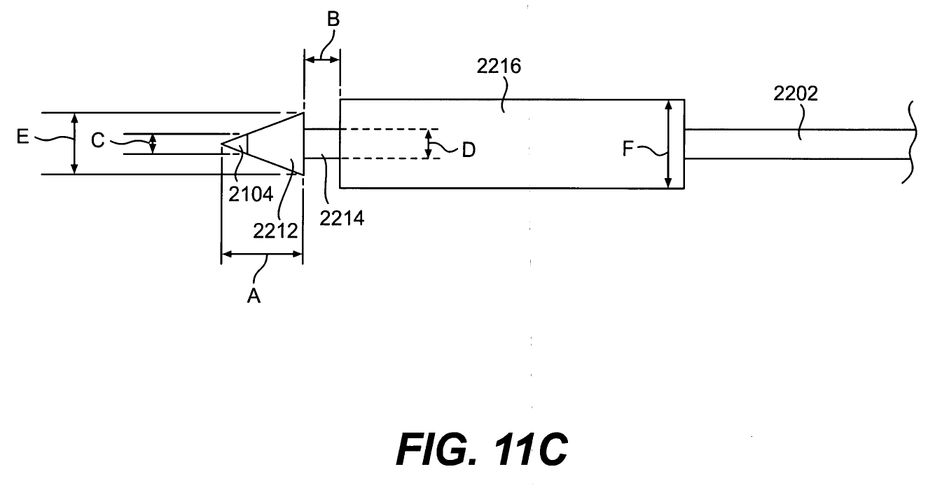

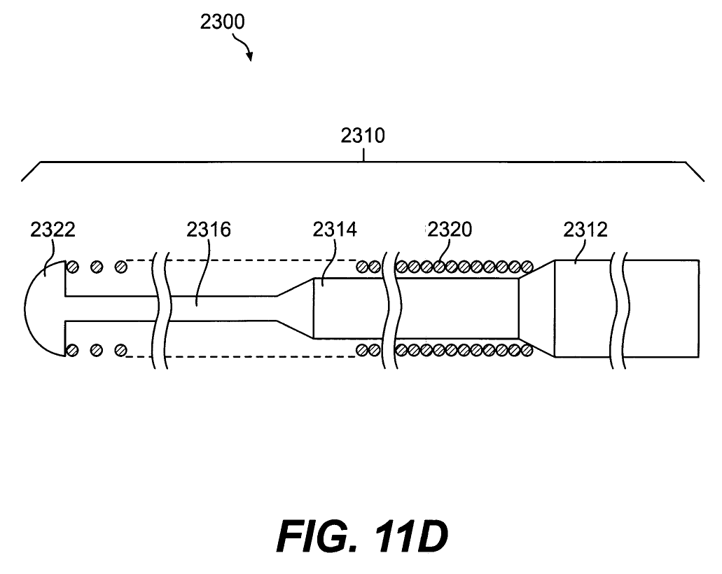

FIGS. 10A-10B and FIGS. 11A-11D illustrate various embodiments of pericardial access devices that may be used to deliver the implantable devices described herein. These access devices provide for less invasive surgical access from a point outside the patient's body, through a transthoracic port (e.g., subxyphoid or intercostal) to the pericardial space around the patient's heart, as will be described in more detail with reference to FIG. 12. A variety of pericardial access devices may be used to deliver the implantable devices described herein, and thus the access devices described hereinafter are shown by way of example, not limitation. Alternative access devices and implant approaches are described in U.S. Published Patent Application No. 2004/0148019 A1 to Vidlund et al., all of which may be utilized in one form or another to deliver the implantable devices described herein.

With specific reference to FIG. 10A, an exemplary embodiment of an access device 1000 is shown. In this exemplary embodiment, access device 1000 includes an outer tube 1100, a securement tube 1200, and a cutter tube 1300. The securement tube 1200 is slidably and coaxially disposed in outer tube 1100, and similarly, the cutter tube 1300 is slidably and coaxially disposed in the securement tube 1200.

Outer tube 1100 may comprise a rigid tubular shaft 1102 formed of stainless steel, for example, having a lumen extending therethrough. A cap 1104 having an interior recess (not visible) may be connected to the distal end of the shaft 1102. A handle 1106 may be connected to a proximal end of the tubular shaft 1102 to facilitate manual manipulation. A vacuum port 1108 may be incorporated into the handle 1106 to facilitate connection to a vacuum source (not shown) for establishing a vacuum in the lumen extending through the tubular shaft 1102.

The securement tube 1200 may comprise a rigid tubular shaft 1202 formed of stainless steel, for example, having a lumen extending therethrough. An annular array of pericardium piercing pins 1204 may be disposed at the distal end of the tubular shaft 1202, and are sized to fit in the recess inside cap 1104 at the distal end of the outer tube 1100 as will be discussed in more detail with reference to FIG. 10B. A handle 1206 may be disposed at the proximal end of the tubular shaft 1202 to facilitate manual manipulation and to act as a stop to prevent the securement tube 1200 from advancing fully into outer tube 1100. A vacuum hole 1208 may be provided through the side of the tubular shaft 1202 to provide a fluid path from the interior of the outer tube 1100 to the interior of the securement tube 1200, thus permitting a vacuum to be established inside the tubular shaft 1202 of the securement tube 1200 by application of a vacuum to vacuum port 1108.

The cutter tube 1300 may comprise a rigid tubular shaft 1302 formed of stainless steel, for example, having a lumen extending therethrough. An annular cutting edge 1304 may be disposed at the distal end of the tubular shaft 1302. An annular ring 1306 may be disposed adjacent the distal end of the tubular shaft 1302 to provide a slidable fluid seal with the inside surface of the tubular shaft 1202 of the securement tube 1200. A series of vacuum holes 1308 may be provided through the side of the tubular shaft 1302 distal of the annular ring 1306 to provide a fluid path from the interior of the securement tube 1200 to the interior of the cutter tube 1300, thus permitting a vacuum to be established inside the tubular shaft 1302 of the cutter tube 1300 by application of a vacuum to vacuum port 1108. A handle 1310 may be disposed at the proximal end of the tubular shaft 1302 to facilitate manual manipulation and to act as a stop to prevent the cutter tube 1300 from advancing fully into securement tube 1200. A visualization device 1320 such as a camera or eye piece 1322 and light source 1324 may be connected to the proximal end of the tubular shaft 1302 to permit direct visualization down the lumen of the cutter tube 1300. Alternatively, an intracardiac echo device may be inserted therethrough, using vacuum for stability, to permit visualization and guidance on the epicardial surface.

With reference to both FIGS. 10A and 10B, the operation of the distal portion of the access device 1000 may be appreciated. The cutter tube 1300 and the securement tube 1200 may be disposed in the outer tube 1100 with the distal ends thereof slightly retracted. The outer tube 1100 may be inserted through a transthoracic port until the distal cap 1104 engages the pericardium (PC) surrounding the heart. Vacuum is applied to port 1108 thus drawing the PC into the lumen of the outer tube 1100, the securement tube 1200, and the cutter tube 1300 to form inward protrusion. The vacuum also draws the PC into the interior recess of the cap 1104 to form an annular fold. The securement tube 1200 may then be advanced distally until the array of pins 1204 passes through the annular fold in the PC, thus mechanically securing and sealing the PC to the access device 1000. The cutter tube 1300 may then be advanced distally until the annular cutting edge 1304 cuts the inward protrusion of the PC, leaving the annular fold of the PC secured to the access device 1000. With the annular fold of the PC mechanically and sealingly connected to the distal end of the access device 1000, and with the outside diameter of the access device 1000 sized to form a seal in the transthoracic port, a sealed access path is established to the pericardial space that is isolated from the pleural space.

With reference to FIGS. 11A-11D, an alternative embodiment of an access device 2000 is shown. Further details and alternative variations of access device 2000 are described in U.S. patent application Ser. No. ______, entitled DEVICES AND METHODS FOR PERICARDIAL ACCESS to Vidlund et al., filed on even date herewith (Attorney Docket No. 07528.0047), the entire disclosure of which is incorporated herein by reference. With reference to FIG. 11A, the alternative access device 2000 includes a stylet member 2100 and a trocar member 2200. Stylet member 2100 is removably insertable into trocar member 2200 as shown in FIG. 11B. The trocar member 2200 includes a tissue grasping portion 2210 that, together with the tip of the stylet member 2100, assists in piercing and retaining the pericardial sac such that it may be pulled away from the heart to enlarge the pericardial space. Once this is accomplished, the stylet member 2100 may be removed from the trocar member 2200 and a guide wire 2300, as shown in FIG. 11D, may be inserted its place. With the guide wire 2300 extending through the trocar member 2200 and into the pericardial space, the trocar member 2200 may be removed leaving the guide wire 2300 in place. The guide wire 2300 thus provides pericardial access from a remote site and may be used to guide and advance delivery devices as described herein.

To illustrate the operation of the tissue grasping portion 2210 of the trocar 2200, it is helpful to consider the environment in which it is particularly suited for use. The pericardial space is defined between the pericardial sac and the epicardial surface of heart. The pericardial sac is very close to (and often in intimate contact with) the epicardial surface of the heart. Therefore, it is helpful to separate the pericardium from the epicardium to provide ready and safe access to the pericardial space. Although separating the pericardium from the epicardium may be readily accomplished using open surgical techniques, it is far more difficult to do so using remote access techniques (e.g., endoscopic, transthorascopic, percutaneous, etc.). To delineate between the epicardial and pericardial layers, the tissue grasping portion 2210 selectively penetrates the pericardial tissue to a limited extent when advanced, and holds onto pericardial tissue when retracted.

More specifically, the tissue grasping portion 2210 is configured to hold onto fibrous tissue such as the pericardium, while not holding onto other less fibrous tissues such as the heart wall (epicardium, myocardium, and endocardium) and surrounding fatty tissues. The tissue grasping portion 2210 is also configured to readily pass through fibrous tissue to a limited, predetermined depth. With this arrangement, the tissue grasping portion 2210 may be advanced to penetrate various layers of fibrous and less-fibrous tissue, stop at a predetermined depth when a fibrous tissue layer is penetrated, and upon retraction, grasp onto the fibrous tissue layer (and not the other less-fibrous layers) to pull the fibrous layer away from the adjacent less-fibrous layer. For example, the access device 2000 may be inserted from a point outside the cardiac space toward the heart, automatically stop when the pericardium is penetrated to a prescribed depth, and selectively hold onto the pericardium when retracted to pull the pericardium away from the epicardial surface, thereby increasing the pericardial space and providing ready access thereto.

As mentioned previously, the access device includes a stylet member 2100 and a trocar member 2200. Stylet member 2100 includes an elongate shaft 2102 having a tissue piercing distal tip 2104 and a proximal hub 2106. Trocar member 2200 includes an elongate hollow shaft 2202, a distally disposed tissue grasping portion 2210 and a proximally disposed hub 2206. The trocar member 2200 includes a lumen extending through the hub 2206, hollow shaft 2202 and distal tissue grasping portion 2210. The elongate shaft 2102 of the stylet member 2100 is insertable into the lumen extending through the trocar member 2200 such that the distal tip 2104 of the stylet device 2100 protrudes from the distal end of the tissue grasping portion 2210 when the proximal hub 2106 of the stylet member 2100 engages and locks with the proximal hub 2206 of the trocar member 2200 as best seen in FIG. 11B. When assembled, the tip 2104 functions integrally with the tissue grasping portion 2210 and may be considered a part thereof.

The tip 2104 of the stylet member 2100 is configured to pierce tissue, particularly fibrous tissue such as the pericardium surrounding the heart, and less fibrous tissue such as the fatty tissues disposed on the exterior of the pericardium. The tip 2104 may be conical with a sharp apex, semi-conical with one or more sharpened edges, or any other geometry suitable for piercing fibrous tissue. Proximal of the apex, the shape of the tip 2104 may be configured to dilate fibrous tissue, such that once the apex pierces the fibrous layer, the tip serves to dilate (as opposed to cut) the hole initiated by the apex. For example, proximal of the apex, the tip 2104 may be circular in cross-section to minimize propagation of the hole initiated by the apex.

A smooth transition may be provided between the tip 2104 of the stylet 2100 and the distal end 2212 of the tissue grasping portion 2210 such that the distal end 2212 continues to dilate the tissue pierced by the apex of the tip 2104. The distal end 2212 may be the same or similar geometry (e.g., conical with a circular cross-section) as the tip 2104 proximal of the apex. A neck 2214 may be provided proximal of the distal end 2212, the profile (e.g., diameter) of which may be selected to allow the fibrous tissue to elastically recoil and resist withdrawal. A shoulder 2216 may be provided proximal of the neck 2214, the profile (e.g., diameter) of which may be selected to limit or stop penetration of the tip 2104 once the shoulder 2216 engages fibrous tissue. Thus, the tip 2104 and distal end 2212 may be configured to penetrate and dilate fibrous tissue, the neck 2214 may be configured to permit elastic recoil of the fibrous tissue and resist withdrawal therefrom, and the shoulder 2216 may be configured to stop penetration through fibrous tissue.

Various sizes and geometries of the aforementioned components are contemplated consistent with the teachings herein. The size and geometry of the tip 2104, and in particular the apex of the tip 2104, may be selected to initially penetrate fibrous tissue (e.g., pericardial tissue) and less-fibrous tissue (e.g., fatty tissue, epicardial tissue, myocardial tissue, etc.). The size and geometry of the tip 2104 proximal of the apex, and the size and geometry of the distal end 2212 may be selected to elastically dilate (but not over-dilate) fibrous tissue initially penetrated by the apex of the tip 2104. The degree of elastic dilation of the fibrous tissue may be sufficiently high to provide for elastic recoil around the neck 2214, but not so high as to cause plastic dilation or tearing of the fibrous tissue. The size and geometry of the neck 2214 may be selected such that the fibrous tissue elastically recoils sufficiently to create a high withdrawal force permitting the fibrous tissue layer to be pulled away from adjacent less-fibrous layers without tearing the fibrous tissue layer. The size and geometry of the shoulder 2216 may be selected such that further penetration is prohibited once the shoulder 2216 engages fibrous tissue.

Taking advantage of the fact that fibrous tissue is tough, tends to elastically deform and tends not to tear, whereas less-fibrous or non-fibrous tissue is weak and tends to plastically deform or tear, the combination of sizes and geometries of the tip 2104, distal portion 2212, neck 2214 and shoulder 2216 may be selected to advance and penetrate through both fibrous and less-fibrous tissue, stop penetration once fibrous tissue is encountered, and grasp the fibrous tissue (while releasing the less-fibrous tissue) upon retraction. As such, the size and geometry of the aforementioned elements may be selected as a function of the characteristics of the tissue layers being separated. In particular, the dimensions and geometries may be chosen to selectively secure (e.g., hold or grasp) tissue of a relatively higher degree of fibrousness or toughness, and release (e.g., not hold or grasp) tissue of a relatively lower degree of fibrousness or toughness.

For selective securing of the pericardium, FIG. 11C and the following Table 1 provides example working dimensions by way of illustration, not limitation. Those skilled in the art will recognize that depending on the tissues layers being separated, these dimensions may be modified according to the teachings herein.

TABLE 1

| | Example | Working | Working |
|---|---|---|---|
| Dimension | Range | Example #1 | Example #2 |
| A | 0.063-0.125″ | 0.125″ | 0.063″ |
| B | 0.020-0.060″ | 0.040″ | 0.020″ |
| C | 0.011-0.020″ | 0.020″ | 0.011″ |
| D | 0.032-0.065″ | 0.032″ | 0.020″ |
| E | 0.032-0.065″ | 0.065″ | 0.032″ |
| F | 0.080-0.100″ | 0.090″ | 0.080″ |

With reference to FIG. 11C and the working examples in Table 1, a number of general observations and statements may be made. For example, after the pericardium is initially pierced by the apex of tip 2104, dimensions A and E are important to achieve the desired amount of elastic pericardial dilation without tearing. Generally speaking, the more pericardial tearing that occurs, the less pericardial retention is achieved. Thus, the larger dimension E is, the longer dimension A may need to be to cause pericardial dilation and minimize tearing. Also, the greater dimension A is relative to E, the lower the force that is required to pierce the pericardium and subsequently dilate it, which may be desirable in some instances. After the pericardium is dilated to the desired degree, the difference between dimensions D and E are important to achieve the desired amount of pericardial retention. To this end, the step from the distal portion 2212 to the neck portion 2214 may be defined as dimension (E-D). Generally speaking, the more elastic pericardial dilation that occurs, the smaller step (E-D) may be to achieve adequate retention. Note also that the depth of tissue penetration is generally governed by the sum of dimensions A and B. While B must be sufficiently wide to accommodate the pericardial layer, dimension A may be adjusted to reduce penetration too far beyond the pericardial layer.

From the foregoing, it is apparent that the tissue grasping portion 2210 together with the tip 2104 of the stylet member 2100 assist in piercing and retaining the pericardial sac such that it may be pulled away from the heart to enlarge the pericardial space. Once this is accomplished, the stylet member 2100 may be removed from the trocar member 2200 and a guide wire 2300, as shown in FIG. 11D, may be inserted in its place. Although a wide variety of guide wire designs may be employed for this purpose, the guide wire design illustrated in FIG. 11D has significant advantages, particularly when used in combination with trocar member 2200.

With continued reference to FIG. 11D, a distal portion of the guide wire 2300 is shown in longitudinal cross-section. Guide wire 2300 includes an elongate shaft 2310 having a proximal end and a distal end. The flexibility of the shaft 2310 increases from its proximal end to its distal end, which may be accomplished by providing reduced diameter or changes in cross section along its length. In the illustrated embodiment, the shaft 2310 of the guide wire 2300 includes a relatively stiff proximal core portion 2312 having a circular cross section, a relatively flexible middle portion 2314 having a rectangular (ribbon-like) cross section, and a highly flexible distal end portion 2316 having a rectangular (ribbon-like) cross section. A radiopaque coil 2320 may be wound around the middle portion 2314 and distal portion 2316, with a proximal end connected to the distal end of the proximal core portion 2312, and a distal end terminating in a distal weld ball 2322 connection to the distal end portion 2316. The distal turns of the coil 2320 may be spaced apart to reduce column strength and increase flexibility as will be discussed in more detail hereinafter.

The guide wire 2300 may be formed of conventional materials using conventional techniques, and may have conventional dimensions except as may be noted herein. The following dimensions are given by way of example, not limitation. The guide wire 2300 may have a diametric profile of about 0.018 inches, for example, or other dimension sized to fit through trocar 2200. In the illustrated embodiment, the proximal core portion 2312 may have a diameter of about 0.018 inches, and the outer profile of the coil 2320 may also have a diameter of about 0.018 inches. The middle portion 2314 may be about 0.010×0.002 inches in cross section, and the distal portion 2316 may be about 0.002×0.004 inches in cross section and about 1.0 inches in length. The guide wire 2300 may have an overall length of about 44.0 inches, for example, or other dimension sized to extend through and beyond the ends of the trocar 2200 and to provide sufficient length for subsequent devices (e.g., sheaths, dilators, balloon catheters, etc.) to be advanced over the wire 2300. The middle 2314 and distal 2316 portions of the guide wire 2300 form an atraumatic section. The middle portion 2314 is highly flexible due to its ribbon-like cross-section and relatively small dimensions. The distal portion 2316 has both high flexibility (due to its ribbon-like cross-section and relatively small dimensions) and low buckle strength (due to the spacing of coil turns). Thus, the middle 2314 and distal 2316 portions are rendered atraumatic. This is particularly true for the distal portion 2316 which is the first portion of the guide wire 2300 to extend beyond the distal end of the trocar 2200 when the guide wire is fully inserted therein. The combination of the loosely spaced coils 2320 and the highly flexible ribbon 2316 allows the distal end of the guide wire to deflect laterally when the it extends out of the distal end of the trocar and engages the heart wall. Because the buckle strength of the highly flexible atraumatic distal portion is less than the force required to penetrate the heart wall (as may occur with stiffer conventional wires), the risk of the guide wire 2300 inadvertently penetrating into the heart wall when advanced through the distal end of the trocar 2200 is minimized.

Exemplary Embodiments of Access and Delivery Methods

In FIG. 12, a transthoracic anterior approach is shown as a dashed line with a distal arrow. This anterior approach may comprise a subxiphoid approach to establish access to the pericardial space, similar to the technique described by Kaplan et al. in U.S. Pat. No. 6,423,051, the entire disclosure of which is incorporated herein by reference. An alternative lateral or posterior approach may utilize similar tools and techniques to access the pericardial space from the side or back between the ribs (intercostal space), similar to the techniques described by Johnson in U.S. Pat. No. 5,306,234, the entire disclosure of which is incorporated herein by reference.

Generally speaking, once pericardial access is established with an access system such as those described with reference to FIGS. 10A-10B or FIGS. 11A-11D, a delivery system such as those described with reference to FIGS. 6-8 may be used to advance and manipulate an implantable device 10/110/210/610/710/910 to the desired deployment position in the pericardial space adjacent the mitral valve MV or a specific part thereof. Assessment of the position and function of the implantable device 10/110/210/610/710/910 relative to internal mitral valve MV structures such as leaflets AL/PL, papillary muscles PM, and regurgitant jet may be performed with ultrasonic imaging such as trans-esophageal, intracardiac or epicardial echocardiography, or x-ray fluoroscopy. These techniques may also be used monitor the adjustment of the size and/or tension of the implantable device 10/110/210/610/710/910 with an adjustment device as described with reference to FIGS. 9A and 9B until the desired acute effect is established. Once in the desired position, the implantable device 10/110/210/610/710/910 may be detached or otherwise disengaged from the distal end of the delivery system, which is subsequently removed.

Detailed Example #1 of Delivery Method

The following detailed example of a delivery method using the delivery system and implant illustrated in FIG. 7 is described by way of example, not limitation, and may be applied to other delivery systems and implants described herein. This method may be broken down into six general steps: (1) establish pericardial access; (2) deliver the first anchor (e.g., near the PDA); (3) deliver the protrusion; (4) deliver the second anchor (e.g., near the LAD); (5) adjust the implant to achieve the desired effect on MV function; and (6) remove the delivery system leaving the implant in place on the outside of the heart.

To establish pericardial access, a needle may be inserted into the chest cavity below the xiphoid as generally shown in FIG. 12. A guide wire (e.g., 0.035″ diameter) may then be inserted into the needle and advanced toward the cardiac space. The needle may then be removed leaving the guide wire in place, and one or more dilators may then be advanced over the guide wire to dilate the percutaneous path. The dilator(s) may then be removed, and the access device illustrated in FIG. 10A may be advanced over the wire adjacent the pericardium. Fluoroscopic visualization (e.g., AP and lateral views) may be used to confirm the desired pericardial access site.

Using the access device illustrated in FIG. 10A, vacuum may be applied to cause the pericardium to be sucked into the distal end thereof, and the tissue piercing pins may be actuated to mechanically secure the pericardium to the access device. The cutter tube may then be advanced to cut and remove a portion of the pericardium in the distal end of the access device, thus establishing a path from the exterior of the body to the pericardial space around the heart.

Initially, the interconnecting member may be loaded into the first anchor and anchor catheter with one side of the interconnecting member extending through the side tube and the other side of the interconnecting member extending through the slotted side tube. Before delivering the anchor, angiographic visualization of the left and/or right coronary arteries may be performed to map the locations of the critical arteries. To deliver the first anchor near the PDA as shown in FIG. 4, for example, the anchor catheter may be manipulated through the access device until the anchor is adjacent the PDA near the last obtuse marginal (OM3), using fluoroscopic visualization to aid navigation. After ascertaining that the first anchor is not positioned over any coronary arteries, vacuum may be applied to the first anchor to temporarily stabilize the anchor on the outside of the heart wall and to pull tissue into the interior of the anchor. The tissue piercing pins may then be actuated to secure the first anchor to the heart wall.

The protrusion may then be advanced along the first anchor catheter by removing one end of the interconnecting member from the slotted tube on the anchor catheter, inserting it through the protrusion and fixing the protrusion midway on the interconnecting member. A delivery tube may be placed about the protrusion to retain it in the delivery configuration, and the delivery tube with the protrusion therein may then be inserted through the access device. By pulling on the opposite end of the interconnecting member and by manipulating the delivery tube, the protrusion may be advanced until it is adjacent the first anchor.

Before delivering the second anchor near the LAD as shown in FIG. 4, the interconnecting member may be inserted into the second anchor and through the side tube of the second anchor catheter. The second anchor may then be slid over the interconnecting member using the anchor catheter, passing through the access device and into the pericardial space. With the aid of fluoroscopic guidance, the second anchor may be positioned next to the junction of the LAD and CFX as seen in FIG. 4, for example. After ascertaining that the second anchor is not positioned over any coronary arteries, vacuum may be applied to the second anchor to temporarily stabilize the anchor on the outside of the heart wall and to pull tissue into the interior of the anchor. The tissue piercing pins may then be actuated to secure the second anchor to the heart wall.

With the first and second anchors secured to the outside of the heart wall, and the protrusion extending therebetween, the interconnecting member may be tightened or cinched using the device illustrated in FIG. 9A, for example. MV function may be simultaneously observed using transesophageal echo (TEE) or intracardiac echo (ICE), and the degree of cinching of the interconnecting member and/or the position of the protrusion may be adjusted to obtain the desired reduction in MV regurgitation (MVR).

With the aid of fluoroscopy, correct anchor positioning may be verified and adequate blood flow may be confirmed in the left coronary arteries. After confirming correct positioning and adequate reduction in MVR, the interconnecting members may be secured by actuating interconnecting member piercing pins with the associated push rods, and the directional catheter shaft may be disconnected from the anchors by actuating the releasable connection with the associated pull wires.

The delivery system may then be removed, and the interconnecting members may be trimmed adjacent the anchors with a cutting device such as an elongate cautery tool. The access device may be removed and the sub-xiphoid access site may be closed using sutures.

Detailed Example #2 of Delivery Method

The following detailed example of a delivery method using the implant and delivery system illustrated in FIGS. 5E and 8A-8D, respectively, is described by way of example, not limitation, and may be applied to other delivery systems and implants described herein. This method may be broken down into six general steps: (1) establish pericardial access; (2) deliver the first (posterior) anchor (e.g., near the PDA); (3) deliver the middle protrusion; (4) deliver the second (anterior) anchor (e.g., near the LAD); (5) adjust the implant to achieve the desired effect on MV function; and (6) remove the delivery system leaving the implant in place on the outside of the heart. The first step of pericardial access may be broken down into a series of sub-steps including: (1.1) percutaneous traversal; (1.2) soft tissue traversal; (1.3) pericardial engagement; (1.4) pericardial traversal; (1.5) pericardial retention; (1.6) pericardial retraction; (1.7) pericardial space access; (1.8) access supplementation; and (1.9) intra-pericardial space navigation. These steps may be taken alone or in a variety of combinations, divisions or repetitions, and the order may be modified as well. Further details and alternative variations of this method are described in U.S. patent application Ser. No. ______, entitled PERICARDIAL ACCESS DEVICES AND METHODS, filed on even date herewith, the entire disclosure of which is incorporated herein by reference.

The sub-steps of (1.1) percutaneous traversal, (1.2) soft tissue traversal, and (1.3) pericardial engagement may be accomplished using conventional tools and techniques modified for this particular application. In a percutaneous method, a needle and wire, and/or blunt dilator and/or introducer may be used to pierce and dilate dermal and soft tissue layers. Alternatively, in a surgical method, a blade and/or coring device and/or cautery device may be used to cut or bore through dermal and soft tissue layers. As a further alternative, a combination of theses tools and methods may be employed for a hybrid percutaneous/surgical methodology. For example, as generally shown in FIG. 12, a small incision may be made in the dermal layers and sub-dermal soft tissue layers just below the xyphoid in the direction of the cardiac space just above the diaphragm (to avoid accessing the pleural space and thus eliminating the need for venting). An introducer sheath (e.g., 8F) and dilator may be inserted through the incised area in a direction toward the inferior-anterior side of the pericardial space, generally coplanar with the annulus of the mitral valve. The desired position of the distal end of the introducer (which may be radiopaque) may be confirmed and/or adjusted using fluoroscopic techniques, and once the introducer is in the desired position, the dilator may be removed therefrom. Thus, the introducer sheath extends across the dermal and soft tissue layers and the distal end thereof resides adjacent the pericardial sac or resides adjacent thereto.

The sub-steps of (1.4) pericardial traversal, (1.5) pericardial retention, (1.6) pericardial retraction, and (1.7) pericardial space access may be accomplished using the system described with reference to FIGS. 11A-11D. For example, with the introducer sheath extending into the chest cavity and its distal end residing adjacent the pericardial sac, and with the dilator having been removed, the access device 2000 (stylet member 2100 and trocar member 2200 assembled) may be inserted into the introducer until the distal tip thereof engages the pericardium. The position of the distal end of the access device (which may be radiopaque) may be confirmed and/or adjusted using fluoroscopic techniques (e.g., AP and lateral views) to ensure the proper pericardial access point and avoid critical coronary structures (e.g., coronary arteries). To further ensure that critical coronary structures such as arteries, veins, etc. are not in the direct path of the access device 2000, fluoroscopic techniques may be employed to illuminate the coronary vasculature and visualize the anticipated path of the access device 2000 relative thereto.

With tactile feedback and fluoroscopic visualization guiding the physician, the access device 2000 may be further advanced until the tip penetrates the pericardial sac and the shoulder engages the outside of the pericardium to stop further penetration. Once the pericardium is penetrated and the shoulder abuts the outside of the pericardial sac, the pericardial layer resides within the neck recess of the access device and is retained therein. The stylet member 2100 may be removed from the trocar member 2200, and a guide wire 2300 may be inserted in its place. While applying gentle proximal traction to the trocar member 2200 to pull the pericardium away from the heart wall, the guide wire 2300 may be advanced until its distal atraumatic end extends beyond the distal end of the trocar 2200 and into the pericardial space. With the guide wire 2300 defining a path extending from a location outside the body, into and partially through the chest cavity, and into the pericardial space, the trocar 2200 and the introducer sheath may be removed therefrom.

The step of (1.8) access supplementation may be accomplished using additional guides, sheaths, dilators, guide wires and/or by a balloon catheter or mechanical dilator advanced over the guide wire. For example, the balloon catheter or dilator may be used to enlarge the size of the hole in the pericardium. A guide catheter (e.g., 6F) may then be advanced over the guide wire into the pericardial space, and the relatively small (0.018 inch diameter) guide wire may be replaced with a relative large (0.035 inch diameter) guide wire. A larger introducer sheath and dilator may then be advanced over the larger guide wire, and the dilator and guide wire may then be removed from the sheath. Thus, the relatively large bore introducer defines a path extending from a location outside the body, into and partially through the chest cavity, and into the pericardial space, thus providing a path for the delivery system described with reference to FIGS. 8A-8D.

The step of (1.9) intra-pericardial space navigation may be accomplished in part by curves provided in the introducer sheath and/or curves provided in the delivery system described with reference to FIGS. 8A-8D. However, the extent of intra-pericardial space navigation may be minimized by the appropriate access approach as shown in FIG. 12. For example, a desirable access approach results in an introducer extending across the right ventricle, over and between the atrial chambers, and toward the left ventricle, with the curve of the introducer sheath generally in coplanar alignment with the mitral valve.

With pericardial access established, the delivery of implantable device 910 may be preceded by angiographic visualization of the left and/or right coronary arteries to map the locations of the critical arteries. The final position of implant 910 may be similar to that generically shown in FIG. 4.

Using fluoroscopic visualization to aid navigation, the first anchor catheter shown in FIG. 8A may be manipulated through the large bore introducer until the first (posterior) anchor is adjacent the PDA near the last obtuse marginal (OM3). After ascertaining that the first anchor is not positioned over any coronary arteries, vacuum may be applied to the first anchor to temporarily stabilize the anchor on the outside of the heart wall and to pull tissue into the interior of the anchor. Alternatively, vacuum may be applied first, and coronary visualization may be used to confirm proper positioning. The bail mechanism may then be actuated as shown in FIGS. 8D(1)-8D(3) to secure the first anchor to the heart wall, and the catheter may be disengaged from the anchor. The catheter may then be removed leaving the interconnecting member as a tether to the first anchor.

The protrusion and associated mid components may then be advanced along the interconnecting member toward the first catheter using the delivery catheter shown in FIG. 8B. The protrusion and associated mid components may be advanced until they abut the first anchor, after which the delivery catheter may be removed leaving the interconnecting member as a tether for the second (anterior) anchor.

The second (anterior) anchor may then be advanced along the interconnecting member toward the protrusion and associated mid components using the second anchor catheter as shown in FIG. 8C. With the aid of fluoroscopic guidance, the second anchor may be positioned next to the bifurcation of the left main artery. After ascertaining that the second anchor is not positioned over any coronary arteries, vacuum may be applied to the second anchor to temporarily stabilize the anchor on the outside of the heart wall and to pull tissue into the interior of the anchor. Alternatively, vacuum may be applied first, and coronary visualization may be used to confirm proper positioning. The bail mechanism may then be actuated to secure the second anchor to the heart wall, and the catheter may be disengaged from the anchor. The catheter may then be removed leaving the interconnecting member as a tether to the second anchor.

With the first and second anchors secured to the outside of the heart wall, and the protrusion extending therebetween, the interconnecting member may be tightened or cinched using the device illustrated in FIG. 9B, for example. MV function may be simultaneously observed using TEE or ICE, and the degree of cinching of the interconnecting member and/or the position of the protrusion may be adjusted to obtain the desired reduction in mitral valve regurgitation (MR). With the aid of fluoroscopy, correct protrusion (and/or anchor) positioning may be verified and adequate blood flow may be confirmed in the left coronary arteries. After confirming correct positioning and adequate reduction in MR, the interconnecting member may be secured to the second anchor by actuating interconnecting member piercing pin with the associated pull wire. The tightening device may be removed, and the interconnecting member may be trimmed adjacent the second anchor with a cutting device such as an elongate cautery tool. The access device may be removed and the sub-xiphoid access site may be closed using sutures.

From the foregoing, it will be apparent to those skilled in the art that the present invention provides, in exemplary non-limiting embodiments, devices and methods for improving the function of a valve (e.g., mitral valve) by positioning an implantable device outside and adjacent the heart wall such that the device applies an inward force against the heart wall or otherwise deforms the heart wall thus acting on the valve to improve leaflet coaptation. Further, those skilled in the art will recognize that the present invention may be manifested in a variety of forms other than the specific embodiments described and contemplated herein. Accordingly, departures in form and detail may be made without departing from the scope and spirit of the present invention as described in the appended claims.

1. A device for securing an implant to body tissue, the device comprising:
a cup defining a chamber and an opening leading to the chamber; and
a tissue piercing mechanism configured to rotate relative to the cup such that the tissue piercing mechanism rotates from a first position wherein the tissue piercing mechanism is substantially within the chamber to a second position wherein the tissue piercing mechanism lies substantially over the opening.

2. The device of claim 1, wherein the chamber includes a suction chamber.

3. The device of claim 1, wherein the tissue piercing mechanism comprises a plurality of tissue piercing pins.

4. The device of claim 3, wherein the pins are curved.

5. The device of claim 4, wherein an inner surface of the cup substantially opposite the opening and defining the chamber is curved such that the inner surface and the pins have substantially the same curvature.

6. The device of claim 3, wherein the tissue piercing mechanism comprises an arm connecting the plurality of tissue piercing pins.

7. The device of claim 6, wherein the arm extends in a direction substantially transverse the plurality of tissue piercing pins.

8. The device of claim 6, wherein the arm is connected to a pivot mechanism configured to rotate the arm and tissue piercing mechanism relative to the cup.

9. The device of claim 1, wherein in the first position, the tissue piercing mechanism is configured so as to allow tissue to enter the chamber.

10. The device of claim 1, wherein in the second position, the tissue piercing mechanism is configured to secure the device to the tissue.

11. The device of claim 1, wherein the tissue piercing mechanism is configured to pierce heart wall tissue.

12. The device of claim 1, wherein the tissue piercing mechanism is configured to be lockable in the second position so as to prevent rotation back to the first position.

13. The device of claim 1, wherein in the first position, a portion of the tissue piercing mechanism is configured to lie adjacent an inner surface of the cup defining the chamber.

14. The device of claim 1, wherein the tissue piercing mechanism is remotely actuatable.

15. The device of claim 1, wherein the tissue piercing mechanism is remotely actuatable via a torque cable.

16. The device of claim 1, further comprising a tissue ingrowth promoting material in cover relation to at least a portion of the device.

17. A device for improving heart valve function, the device comprising:
an elongate member having a first end and a second end; and
an anchoring member associated with each of the first end and the second end and configured to secure the device relative to the heart,
wherein each of the anchoring members comprises
a cup defining a chamber and an opening leading to the chamber; and
a tissue piercing mechanism configured to rotate relative to the cup such that the tissue piercing mechanism rotates from a first position wherein the tissue piercing mechanism is substantially within the chamber to a second position wherein the tissue piercing mechanism lies substantially over the opening.

18. The device of claim 17, wherein the tissue piercing mechanism includes a plurality of tissue piercing pins.

19. The device of claim 17, wherein the tissue piercing mechanism is remotely actuatable.

20. The device of claim 19, wherein the tissue piercing mechanism is remotely actuatable via a torque cable.

21. The device of claim 17, further comprising a protrusion disposed between the anchoring members and configured to be disposed adjacent an external surface of a heart wall when the device is secured with respect to the heart.

22. The device of claim 21, wherein the protrusion is configured to exert an inward force on the heart wall proximate a valve when the device is secured relative to the heart, the inward force being sufficient to alter a function of the valve.

23. The device of claim 17, wherein the chambers are suction chambers.

24-36. (canceled)

37. A delivery system for delivering an implant to a heart, the delivery system comprising:
a first catheter configured to simultaneously deliver to the heart an elongate member and a first anchor mechanism attached to a first end of the elongate member;
a second catheter configured to advance an intermediate component along the elongate member until the intermediate component is adjacent the first anchor mechanism; and
a third catheter configured to advance a second anchor mechanism along the elongate member to a position adjacent the intermediate component and on a side of the intermediate component opposite the first anchor mechanism.

38. The delivery system of claim 37, wherein the first catheter comprises a tube defining a lumen configured to receive the elongate member and a shaft configured to releasably connect to the first anchor mechanism.

39. The delivery system of claim 38, wherein the shaft defines a suction lumen.

40. The delivery system of claim 39, wherein the suction lumen is configured to be placed in fluid communication with a chamber defined by the first anchor mechanism.

41. The delivery system of claim 38, wherein the tube comprises a slit along a length of the tube configured to permit removal of the elongate member from the tube lumen.

42. The delivery system of claim 37, wherein the first catheter comprises an actuator for releasing the first anchor mechanism.

43. The delivery system of claim 37, wherein the first catheter comprises an actuator configured to actuate the first anchor mechanism such that it becomes secured to the heart.

44. The delivery system of claim 37, wherein the third catheter comprises a tube defining a lumen and a shaft configured to releasably connect to the second anchor mechanism.

45. The delivery system of claim 44, wherein the shaft defines a suction lumen.

46. The delivery system of claim 45, wherein the suction lumen is configured to be placed in fluid communication with a chamber defined by the second anchor mechanism.

47. The delivery system of claim 44, wherein the tube comprises a slit along a length of the tube.

48. The delivery system of claim 37, wherein the third catheter comprises an actuator for releasing the second anchor mechanism.

49. The delivery system of claim 37, wherein the third catheter comprises an actuator configured to actuate the second anchor mechanism such that it becomes secured to the heart.

50. The delivery system of claim 37, wherein the second catheter comprises a shaft having a distal end configured to abut the intermediate component so as to push the intermediate component along the elongate member.

51. The delivery system of claim 50, wherein the shaft defines a lumen configured to receive the elongate member.

52. The delivery system of claim 37, further comprising a tightening device for adjusting a length of the elongate member between the first and second anchor mechanisms when implanted in the heart.

53. The delivery system of claim 52, wherein the tightening device comprises an actuator to actuate a securing member to secure the second anchor mechanism to the elongate member.

54. The delivery system of claim 37, further comprising an access device for providing access to the pericardial space.

55. The delivery system of claim 37, wherein the system is configured to access the pericardial space so as to deliver the implant to the pericardial space.

56. The delivery system of claim 37, wherein the system is configured to deliver an implant configured to treat a heart valve.

57. A device for improving heart valve function, the device comprising:
an elongate member having a first end and a second end;
an anchoring member associated with each of the first end and the second end and configured to secure the device relative to the heart such that the device provides a compressive force to an exterior portion of the heart sufficient to alter valve function; and
an intermediate component comprising a sleeve configured to be advanced over the elongate member and to be positioned between each anchoring member when the device is implanted in the heart, the sleeve being configured to distribute the force applied by the elongate member to the heart.

58. The device of claim 57, wherein the sleeve comprises expanded polytetrafluoroethylene (ePTFE).

59. The device of claim 57, wherein the intermediate component comprises two sleeves.

60. The device of claim 59, wherein the intermediate component comprises a protrusion configured to be positioned between the two sleeves.

61. The device of claim 60, wherein the protrusion is configured to at least partially provide the compressive force sufficient to alter valve function.

62. The device of claim 57, wherein the intermediate component comprises a protrusion, the protrusion being configured to at least partially provide the compressive force sufficient to alter valve function.

63. The device of claim 57, wherein the sleeve is deformable.

64. The device of claim 57, wherein the sleeve is configured to conform to coronary vasculature.

65. The device of claim 57, wherein the sleeve is a tubular sleeve.

66. The device of claim 62, wherein the height of the sleeve approximates the height of the protrusion as measured in a direction substantially transverse to the elongate member.

67-83. (canceled)

84. A system for treating a heart valve, the system comprising:
an access device configured to access the pericardial space from a remote location, a portion of the access device being configured to be automatically inserted through the pericardium to a predetermined depth beyond the pericardium and to separate the pericardium from the epicardium; and
an implant configured to be delivered to the pericardial space and to be secured relative to the heart so as to exert a compressive force on the heart sufficient to alter valve function.

85. The system of claim 84, wherein the access device is configured to separate the pericardium from the epicardium without the use of suction.

86. The system of claim 84, wherein the portion of the access device includes a distal portion of the access device.

87. The system of claim 86, wherein the distal portion comprises a dilation member and a region of reduced cross-section proximal the dilation member.

88. The system of claim 87, wherein the access device includes a shoulder proximal the region of reduced cross-section, the shoulder being configured to abut the pericardium such that the shoulder cannot be inserted past the pericardium.

89. The system of claim 87, wherein the access device is configured such that once the dilation member is inserted past the pericardium, moving the access device in a proximal direction causes the dilation member to separate the pericardium from the epicardium.

90. The system of claim 87, wherein the dilation member is configured to elastically dilate the pericardium.

91. The system of claim 90, wherein the dilation member is configured to elastically dilate the pericardium by an amount such that the dilation member can separate the pericardium from the epicardium without tearing the pericardium upon moving the access device in a proximal direction.

92. The system of claim 87, wherein the dilation member is configured to dilate the pericardium during insertion by an amount such that the pericardium elastically recoils around the region of reduced cross-section.

93. The system of claim 84, further comprising a dilating tool configured to dilate the pericardium prior to delivering the implant to the pericardial space.

94. The system of claim 84, wherein the portion of the access device includes a region of enlarged cross-section configured to engage the pericardium to separate the pericardium from the epicardium upon moving the portion in a proximal direction.

95. The system of claim 84, further comprising a stylet configured to pierce the pericardium.

96. The system of claim 95, wherein the access device includes a trocar and the stylet is configured to carry the trocar during insertion of the trocar through the pericardium.

97. The system of claim 84, wherein the access device includes a trocar and the system further comprises a stylet configured to carry the trocar during insertion of the trocar through the pericardium.

98. The system of claim 97, wherein the stylet is configured to be removed from the trocar prior to delivering the implant.

99. The system of claim 84, further comprising a guidewire configured to be delivered to the pericardial space via the access device.

100. The system of claim 99, wherein the guidewire is configured to deliver the implant to the pericardial space.
